SEQUENCE LISTING (13 sequences)

SEQ ID NO: 1 - DNA, 36 bp, Artificial Sequence
  Description of Artificial Sequence Synthetic oligonucleotide
tgggatgattgattactcccagctgtcgtatgccgt

SEQ ID NO: 2 - DNA, 20 bp, Artificial Sequence
  Description of Artificial Sequence Synthetic probe
  modified_base (6)..(16): a, c, t, g, unknown or other
atgatnnnnnnnnnnnctag

SEQ ID NO: 3 - DNA, 20 bp, Artificial Sequence
  Description of Artificial Sequence Synthetic probe
atgatacaccgaggctctag

SEQ ID NO: 4 - DNA, 20 bp, Artificial Sequence
  Description of Artificial Sequence Synthetic probe
  modified_base (4)..(8): a, c, t, g, unknown or other
  modified_base (13)..(14): a, c, t, g, unknown or other
  modified_base (17)..(19): a, c, t, g, unknown or other
tggnnnnntaccnntannna

SEQ ID NO: 5 - DNA, 20 bp, Artificial Sequence
  Description of Artificial Sequence Synthetic probe
tggaccgatacccctagata

SEQ ID NO: 6 - DNA, 13 bp, Artificial Sequence
  Description of Artificial Sequence Synthetic probe
tcagactattggc

SEQ ID NO: 7 - DNA, 13 bp, Artificial Sequence
  Description of Artificial Sequence Synthetic probe
tctgaagggatgc

SEQ ID NO: 8 - DNA, 13 bp, Artificial Sequence
  Description of Artificial Sequence Synthetic probe
gccgctcgaaatc

SEQ ID NO: 9 - DNA, 13 bp, Artificial Sequence
  Description of Artificial Sequence Synthetic probe
cttgggtctcggc

SEQ ID NO: 10 - DNA, 13 bp, Artificial Sequence
  Description of Artificial Sequence Synthetic probe
cttccggtcggtc

SEQ ID NO: 11 - DNA, 20 bp, Artificial Sequence
  Description of Artificial Sequence Synthetic probe
gttatgattacacgtgttcc

SEQ ID NO: 12 - RNA, 20 nt, Artificial Sequence
  Description of Artificial Sequence Synthetic probe
guuaugauuacacguguucc

SEQ ID NO: 13 - protein, 20 aa, Artificial Sequence
  Description of Artificial Sequence Synthetic probe
IFFPDDYLAINLSMCFLGVF

(19) United States
(12) Patent Application Publication
(10) Pub. No.: US 2021/0407624 A1
(43) Pub. Date: Dec. 30, 2021

(54) SYSTEMS AND METHODS FOR ANALYZING SEQUENCING DATA

(71) Applicant: THE TRUSTEES OF COLUMBIA UNIVERSITY IN THE CITY OF NEW YORK, New York, NY (US)

(72) Inventors: Harmen H. BUSSEMAKER, New York, NY (US); Chaitanya RASTOGI, New York, NY (US); Hans Tomas RUBE, New York, NY (US)

(73) Assignee: THE TRUSTEES OF COLUMBIA UNIVERSITY IN THE CITY OF NEW YORK, New York, NY (US)

(21) Appl. No.: 17/476,113

(22) Filed: Sep. 15, 2021

Related U.S. Application Data
(63) Continuation of application No. PCT/US2020/023017, filed on Mar. 16, 2020.
(60) Provisional application No. 62/870,226, filed on Jul. 3, 2019.
(60) Provisional application No. 62/827,643, filed on Apr. 1, 2019.
(60) Provisional application No. 62/819,311, filed on Mar. 15, 2019.

(51) Int. Cl. G16B 35/20; G16B 30/00; G16B 20/00

(52) U.S. Cl. CPC G16B 35/20; G16B 20/00; G16B 30/00

(57) ABSTRACT

The disclosed subject matter provides systems and methods for identifying bioactivities of biopolymers from sequence data of the biopolymers. The disclosed system can include a processor configured to receive the input data and a storage medium including instructions operable when executed by the processors. The instructions can cause the system to obtain the input data and generate an evaluative model configured to acquire a biophysical model parameter, a model interaction parameter, a count table parameter, or combinations thereof utilizing the input data. The evaluative model can be configured to simultaneously use multiple biophysical models to represent one or more sequence recognition modes of the biopolymers, evaluate the biopolymers using the evaluative model, and generate a value using the evaluating model that corresponds to the bioactivity of each biopolymer.

### CROSS-REFERENCE TO RELATED APPLICATION

This application is a continuation of International Patent Application No. PCT/US 2020/023017 filed Mar. 16, 2020, which claims priority to U.S. Provisional Patent Applications Nos. 62/819,311, which was filed on Mar. 15, 2019, 62/827,643, which was filed on Apr. 1, 2019, and 62/870,226, which was filed on Jul. 3, 2019, the entire contents of which are incorporated by reference herein.

### STATEMENT REGARDING FEDERALLY SPONSORED RESEARCH

This invention was made with government support under HG003008 and GM082797 awarded by the National Institutes of Health. The government has certain rights in the invention.

### SEQUENCE LISTING

This application contains a Sequence Listing, which has been submitted in ASCII format via EFS-Web, and is hereby incorporated by reference in its entirety. The ASCII copy, created on Apr. 9, 2020, is named 070050_6355_SL.TXT and is 3,756 bytes in size.

### BACKGROUND

MEME Suite, Hidden Markov Models, Gibbs Sampler, ChIPMunk, and HOMER can be considered traditional ‘probabilistic’ techniques to detect and characterize base preference patterns in sets of unaligned DNA sequences. In theory, there is no limit to the size of the motifs (short reoccurring sequences) that can be discovered using these approaches. In practice, however, they can fail to allow for the building of accurate quantitative models from high-throughput functional genomics data.

For instance, certain approaches used to evaluate high-throughput functional genomic data utilize a penalty associated with base substitutions away from the optimal sequence. In such approaches, the penalty associated with the existing algorithms can be tightly correlated to the criterion that was used to define the set of ‘bound’ sequences. Such tight binding can be problematic. DNA-binding specificity is a quantitative property of the transcription factor (TF) proteins, where estimates should not depend on how a training set of DNA sequences was defined. Furthermore, such approaches can fail to provide accurate quantitative models for measuring the interaction strength/activity between a target molecule and other members of a class of semi-random, DNA/RNA-templated molecules.

Other approaches using evaluative models have respective shortcomings that cause a gap in the functionality present in the art. For instance, while the evaluative program DeepBind performs well on classification tasks involving the identification of sequences bound by transcription factors, the models underlying its functionality can be unable to quantitatively characterize protein-DNA binding affinity or address low-affinity binding sites. In addition, DeepBind models can be difficult to interpret, owing to the complexity of training provided by certain deep neural networks.

Accordingly, there remains a need for techniques that improve upon the drawbacks of these approaches for analyzing sequence data.

### SUMMARY

The disclosed subject matter provides systems and methods for identifying bioactivities of biopolymers from sequence data of the biopolymers.

An example system can include a processor configured to receive the input data and a storage medium including instructions operable when executed by the processors. The instructions can cause the system to obtain the input data and generate an evaluative model configured to acquire a biophysical model parameter, a model interaction parameter, a count table parameter, or combinations thereof utilizing the input data. The evaluative model can be configured to simultaneously use multiple biophysical models to represent one or more sequence recognition modes of the biopolymers, evaluate the biopolymers using the evaluative model, and generate a value using the evaluating model that corresponds to the bioactivity of each biopolymer.

In certain embodiments, the biopolymers can include a biopolymer library. In non-limiting embodiments, the biopolymer library can include a single-stranded deoxyribonucleic acid (DNA), double-stranded DNA, DNA with synthetic bases, DNA with unnatural base pairings, ribonucleic acid (RNA), RNA with synthetic bases, a protein with natural amino acids, a protein with unnatural amino acids, genomic DNA, methylated DNA, a fragment of genomic DNA, a plasmid, or combinations thereof.

In certain embodiments, the system can further include an analytic platform that is configured to generate input data corresponding to the biopolymers.

In certain embodiments, the input data can include at least two sets of biopolymer sequences. The at least two sets of biopolymer sequences can include a first set of biopolymer sequences and a second set of biopolymer sequences data corresponding to a sequence of a biopolymer generated in the different conditions. In non-limiting embodiments, the different conditions can include an environmental condition, a disease state, a cell type or state, a tissue, a genotype, the presence or absence of a specific molecular target, the chemical modification status of the biopolymers (such as methylation), or a combination thereof.

In certain embodiments, the input data can be compiled into a count table. The count table can include a record of sequences of the biopolymers and a number of times that a certain biopolymer or “probe,” is observed in the different conditions.

In certain embodiments, the input data are stored in the storage medium.

In certain embodiments, the evaluative model is optimized from at least one function representing a statistical distribution of the input data, a selection rate for each sequence of the input data, a binding affinity of the biopolymers, bioactivity of the biopolymers, an environmental condition of the biopolymers, or combinations thereof.

In certain embodiments, the evaluative model can be configured to generate the value corresponding to the log-likelihood (i.e., the natural logarithm of the likelihood) using a sum of generalized Poisson log-likelihood functions over the count table. In non-limiting embodiments, the sum of generalized Poisson log-likelihood functions over the count table can be calculated based on sequencing depth, a probe bias in the input data, and a selection function. The sequencing depth, the probe bias in the input data, and the selection function can be adjusted based on a target value to generate. In certain embodiments, the target value can be binding affinity, binding free energy, kinetic rate, and combinations thereof.

In non-limiting embodiments, the evaluative model can be used to identify at least one Michaelis constant (K_{M}), dissociation constant (K_{d}), a presence of a putative binding site, a functional effect of single-nucleotide polymorphism (SNP), a transcription factor activity, a structural feature of a transcription factor, an immune response to a pathogen, thermostability, pH stability, protein binding strength, an enzymatic activity, a biopolymer interaction, antibiotic resistance, a difference between healthy and diseased cells, a cellular response to environmental variations, a regulatory pathway, an ability to penetrate a cell or tissue, or combinations thereof.

In certain embodiments, the disclosed system can further include an output device configured to display the generated value.

The disclosed subject matter also provides methods for identifying bioactivities of biopolymers from sequence data of the biopolymers. An example method can include obtaining input data corresponding to the biopolymers and generating an evaluative model utilizing the input data, where the evaluative model is configured to acquire a biophysical model parameter, a model interaction parameter, a count table parameter, or combinations thereof from the input data. The evaluative model can be configured to simultaneously use multiple biophysical models to represent one or more sequence recognition modes of biopolymers. The method can also include evaluating the biopolymers using the evaluative model and generating a value using the evaluating model that corresponds to the bioactivity of each biopolymer.

In certain embodiments, the method can further include obtaining the biopolymers, obtaining a first set of sequence data corresponding to sequence data for at least one of the biopolymers, exposing the biopolymers to a predetermined condition, obtaining a second set of sequence data corresponding to sequence data that for the biopolymers in the predetermined condition, and generating at least the first and second sets of sequence data as the input data for the evaluative model.

In certain embodiments, the method can further include compiling the input data into a count table, where the count table includes a record of sequences of the biopolymers and a number of times that a certain biopolymer of the biopolymer library, or a probe, is observed in an experimental condition.

In certain embodiments, the method can further include optimizing the evaluative model using at least one function representing a statistical distribution of the input data, a selection rate for each sequence of the input data, a binding affinity of the biopolymers, bioactivity of the biopolymers, an environmental condition of the biopolymers, or combinations thereof.

In non-limiting embodiments, the disclosed method can be used to identify at least one Michaelis constant (K_{M}), dissociation constant (K_{d}), a presence of a putative binding site, a functional effect of single-nucleotide polymorphism (SNP), a transcription factor activity, a structural feature of a transcription factor, an immune response to a pathogen, thermostability, pH stability, protein binding strength, an enzymatic activity, a biopolymer interaction, antibiotic resistance, a difference between healthy and diseased cells, a cellular response to environmental variations, a regulatory pathway, an ability to penetrate a cell or tissue, or combinations thereof.

### BRIEF DESCRIPTION OF THE DRAWINGS

Further features and advantages of the present disclosure will become apparent from the following detailed description taken in conjunction with the accompanying figures showing illustrative embodiments of the present disclosure, in which:

FIG. 1 is a block diagram illustrating one or more elements of the presently disclosed system.

FIG. 2 is a flow diagram of exemplary methods of the presently disclosed subject matter.

FIG. 3 is a diagram providing an exemplary structure of a library in accordance with the disclosed subject matter.

FIG. 4 is a diagram providing exemplary data types in accordance with the disclosed subject matter.

FIG. 5 is a diagram providing exemplary constructions of the experiment in accordance with the disclosed subject matter.

FIG. 6 is a flow diagram providing an exemplary structure of the experiment in accordance with the disclosed subject matter.

FIG. 7 is a diagram providing an exemplary count table in accordance with the disclosed subject matter.

FIG. 8 is a flow diagram providing further elements of the specific aspects of the evaluative model in accordance with the disclosed subject matter.

FIG. 9 is a diagram providing further elements of the specific aspects of the evaluative model in accordance with the disclosed subject matter.

FIG. 10 is a diagram providing further elements of the specific aspects of the evaluative model in accordance with the disclosed subject matter.

FIG. 11 is a diagram providing further elements of the specific aspects of the evaluative model in accordance with the disclosed subject matter.

FIG. 12 is a flow diagram illustrating exemplary methods of the presently disclosed subject matter.

FIG. 13 illustrates the utilization of the disclosed evaluative model described herein.

FIG. 14 illustrates the utilization of the disclosed evaluative model described herein.

FIG. 15 illustrates the utilization of the disclosed evaluative model described herein.

FIG. 16 illustrates the utilization of the disclosed evaluative model described herein.

FIG. 17 illustrates the utilization of the disclosed evaluative model described herein.

FIG. 18 illustrates the utilization of the disclosed evaluative model described herein.

FIG. 19 illustrates the utilization of the disclosed evaluative model described herein.

FIG. 20 illustrates the utilization of the disclosed evaluative model described herein.

FIG. 21 illustrates the utilization of the disclosed evaluative model described herein.

FIG. 22 illustrates the utilization of the disclosed evaluative model described herein.

### DETAILED DESCRIPTION

The presently disclosed subject matter provides techniques for analyzing sequencing data. As embodied herein, the present disclosure provides systems and methods for identifying bioactivities of biopolymers from sequence data of the biopolymers. The bioactivity can include binding affinity, binding free energy, interaction strength, kinetic rate, enzyme activity, antibiotic resistance, thermostability, a difference between healthy and diseased cells, cellular response to environmental variations, a regulatory pathway, an ability to penetrate a cell or tissue, a differential condition of biopolymers, or combinations thereof.

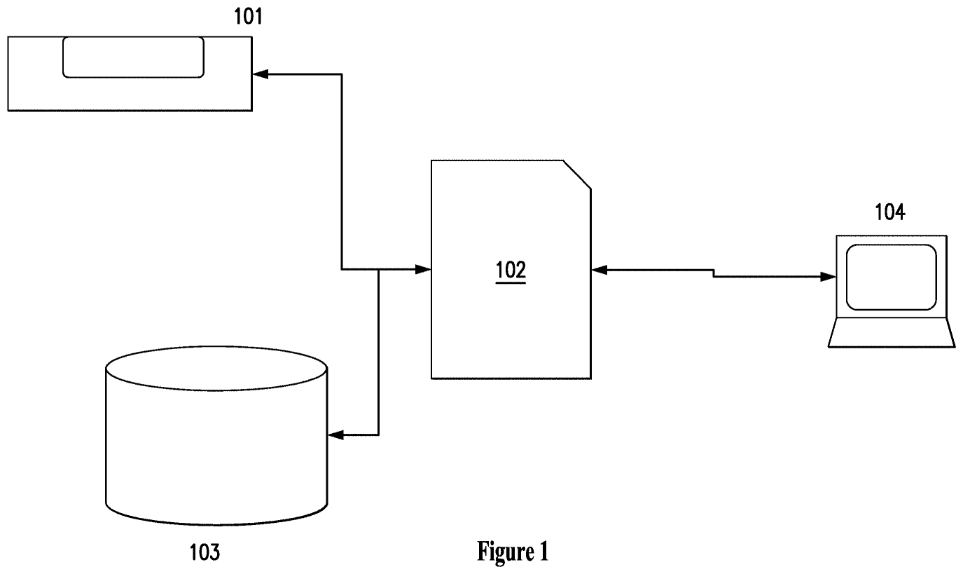

With reference to FIG. 1, the disclosed system can include a data analytic platform 101, a processor 102, a storage device 103, and an output device 104. The storage device 108 can be coupled to the processor 102 and include instructions operable when executed by the processors. The instructions can cause the system to obtain the input data from the analytic platform and generate an evaluative model configured to acquire a biophysical model parameter, a model interaction parameter, a count table parameter, or combinations thereof utilizing the input data. The evaluative model can be configured to simultaneously use multiple biophysical models to represent one or more sequence recognition modes throughout which the biopolymers interact with the target molecule, evaluate the biopolymer library using the evaluative model, and generate a value using the evaluating model that corresponds to the bioactivity of biopolymer.

The input data can be obtained from the data analytic platform 101. This processed data is passed, directly or indirectly, to the processor 102. The present systems, method, and computer products can be implemented by hardware and software, that, in one or more operative collections, configurations or arrangements, permit the generation or access of one or more analytical or evaluative models and the utilization thereof. For example, the disclosed systems can include a processor 102 that can be configured by one or more modules, such as code executing in a processor to generate an evaluative model for identifying coefficient parameters of a DNA/RNA/protein sequence recognition model using data obtained from one or more data analytic platform(s) 101. In non-limiting embodiments, the disclosed system can further include a remote storage 104 and the output device 105.

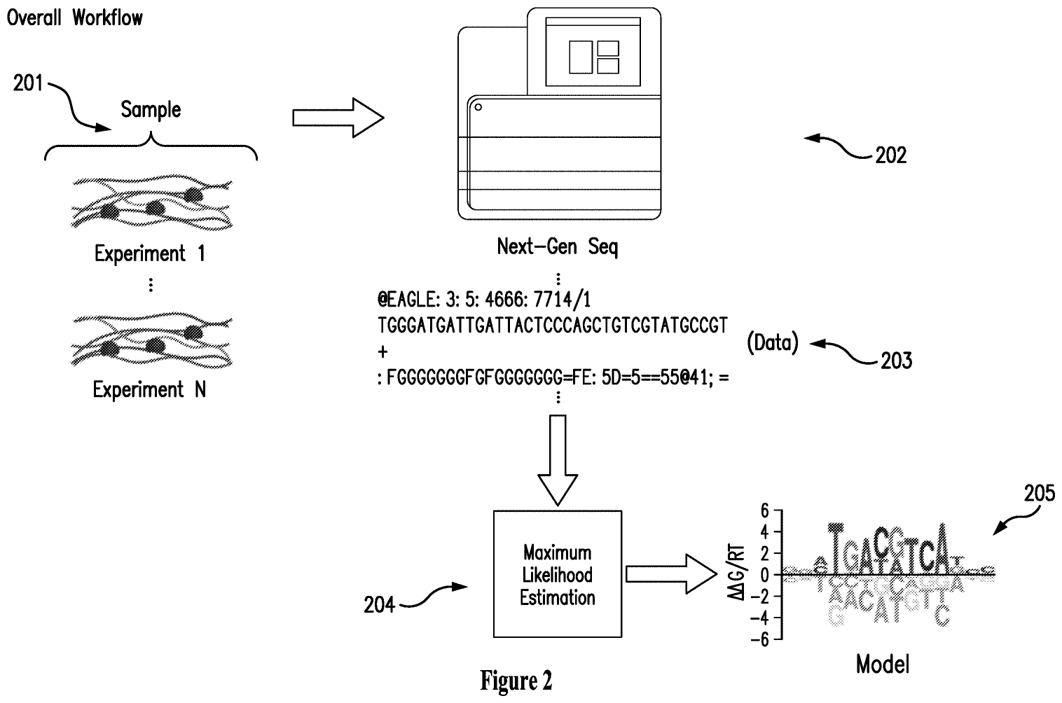

FIG. 2 provides an exemplary diagram of a method for determining bioactivities of biopolymers from sequence data. As shown, assay results 201 can be acquired from at least one experiment. The assay results can be produced by existing assays, which use

DNA/RNA fragments directly or indirectly (e.g., DNA-templated transcription and translation, in vitro or in vivo). The results can be provided to a sequencer 202, which can be a part of the overall data analytic platform 101. The sequencer 202 can be configured to generate input data 203 that corresponds to a biopolymer library of the assay results 201 provided to the sequencer 202. Those possessing an ordinary level of skill in the requisite art can appreciate that other sequence data obtained from various high-throughput assays can be used in the manner and for the purposes described herein.

The input data 203 can be used by one or more processors or computers to generate an evaluative model that identifies coefficient parameters of a DNA/RNA/protein sequence recognition model from the input data. In non-limiting embodiments, the model generated by the processor or computer 102 can include a biophysical model of DNA binding affinity where the relevant parameter set has been optimized using maximum likelihood estimation (MLE) techniques 204. The resulting model 205 can be used to probe sequences and determine the likely location of protein binding sites and other biologically useful information.

In certain embodiments, the input data can include at least two sets of biopolymer sequences. For example, the input data can include a first set of biopolymer sequence data and a second set of biopolymer sequence data corresponding to a sequence of a biopolymer generated in the different conditions. For example, the input data can be obtained by obtaining a first set of sequence data corresponding to sequence data for at least one of the biopolymers and exposing the biopolymers to a predetermined condition. Then a second set of sequence data corresponding to sequence data that for the biopolymers in the predetermined condition can be obtained for generating at least the first and second sets of sequence data as the input data for the evaluative model. In non-limiting embodiments, the different conditions can include an environmental condition, a disease state, a cell type or state, a tissue, a genotype, the presence or absence of a specific molecular target, the chemical modification status of the biopolymers (such as methylation), or a combination thereof.

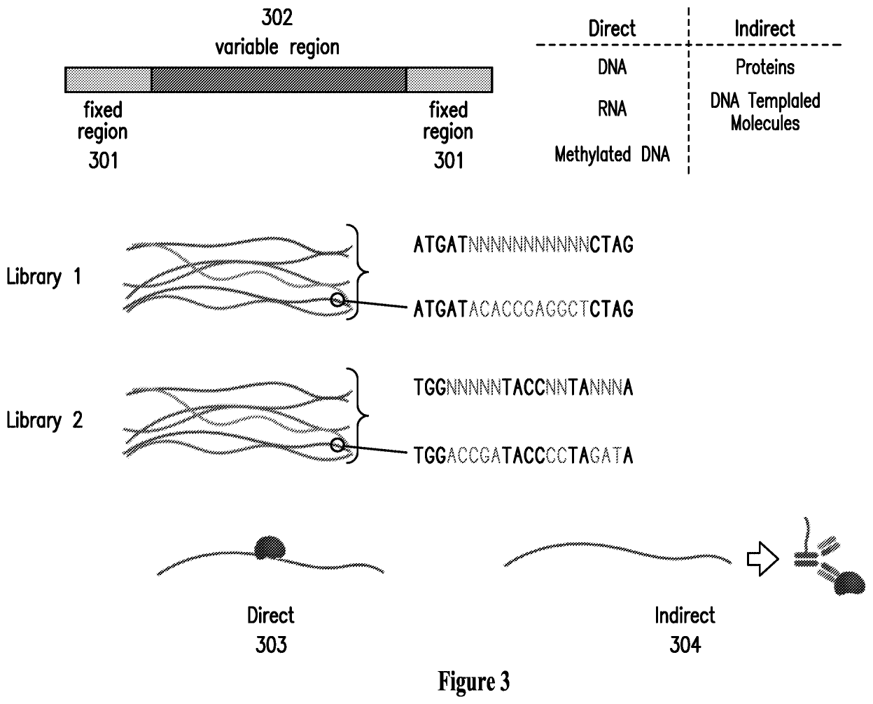

In certain embodiments, the disclosed subject matter provides methods of obtaining input data, wherein the input data can comprise at least two sequencing libraries (FIG. 3). As used herein, the term “library” refers to a pool of deoxyribonucleic acid (DNA) or ribonucleic acid (RNA) fragments. In non-limiting embodiments, the library can also include a pool of DNA and RNA fragments that can attach to or be located inside of a virus or a cell. The DNA and RNA fragments can be a probe for various genomics or molecular biology experiments. In certain embodiments, the library can be a random synthetic library. The random library can include multiple DNA/RNA fragments, which can be a probe for various genomics and/or molecular biology experiments. As shown in FIG. 3, the probe can include a fixed sequence 301, a variable sequence 302, or a combination thereof. Variable DNA/RNA sequence can be a random sequence, a custom-designed sequence, a sampled sequence from a genome. In non-limiting embodiments, the library can be constructed with synthetic bases, unnatural amino acids, unnatural base pairing, or combinations thereof. In non-limiting embodiments, the library can interact with a target directly or indirectly. When used directly 303, the DNA/RNA fragments can interact with the target in the predetermined experiment. The DNA/RNA fragments can also be consumed or modified by the target. When used indirectly 304, the DNA/RNA fragments can be used to template molecules that interact with or are consumed by the target in the experiment.

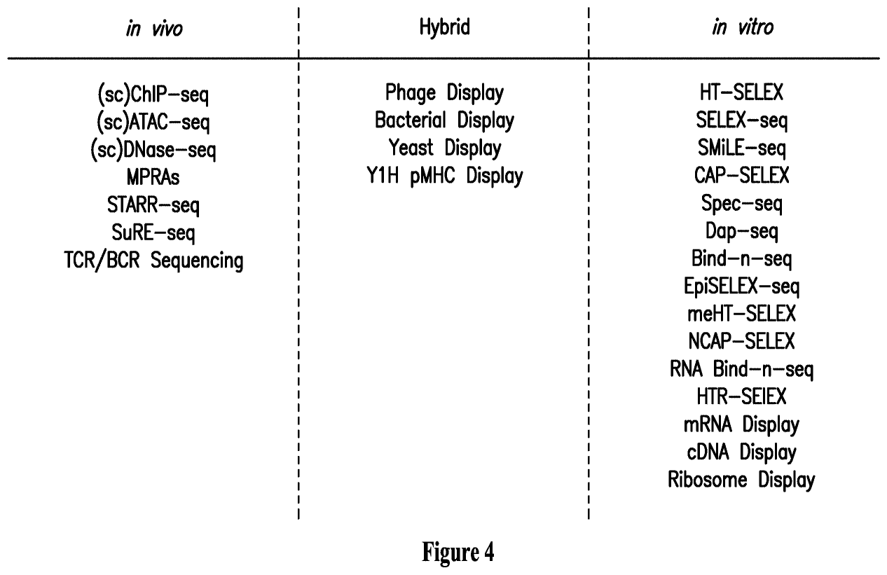

As shown in FIG. 4, certain sequencing technology can be used on a massively parallel scale to probe in a variety of ways. For example, the sequencing technology can include the in vitro profiling of molecular interactions (e.g., HT-SELEX, SELEX-seq, SMiLE-seq, CAP-SELEX, SPEC-seq, DAP-seq, Bind-n-seq, meHT-SELEX, NCAP-SELEX, RNA Bind-n-seq, HTR-SELEX, mRNA display, cDNA display, and Ribosome display assays), the in vivo profiling of molecular interactions (e.g., ChIP-seq, ATAC-seq, DNase-seq, and related methods), gene expression profiling (e.g., RNA-seq), massively parallel reporter assays (e.g., MPRA, STAR-seq, or SuRE-seq), 3D chromatin interaction analysis (e.g., Hi-C or SPRITE), deep-sequencing of immune receptor repertoires (TCR/BCR-seq), and the hybrid profiling of molecular interactions (e.g., phage display, bacterial display, yeast display, and Y1H pMHC display assays). As used herein, the hybrid profiling can refer to an in vitro profiling performed with cells as a medium that can generate variants. In non-limiting embodiments, the disclosed assays can be applied to analyze at least two libraries. In certain embodiments, a large number of libraries can be processed simultaneously.

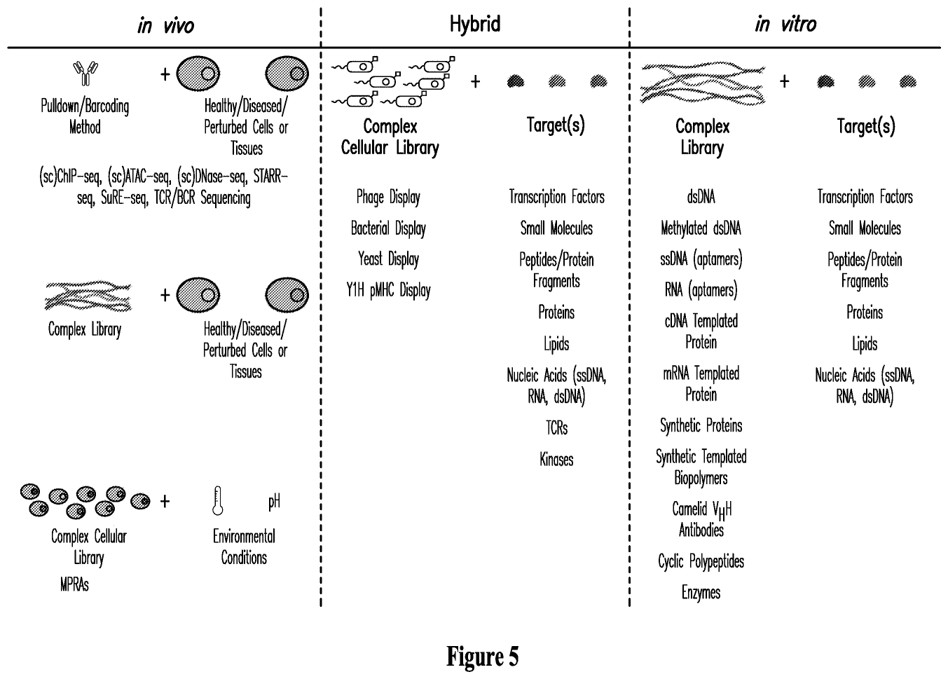

As used herein, the term “experiment” refers to an attempt at measuring or observing the molecular interaction between one or more molecules under certain conditions. In certain embodiments, each experiment can be constructed with a predetermined design and provide a set of sequencing libraries. The predetermined design can include in vivo, in vitro, and hybrid designs (FIG. 5). As used herein, the experiment with the hybrid design can refer to an in vitro experiment designed with cells, which can be a medium that can generate variants. For example, hybrid experiments can be constructed to identify an interaction between a cellular library and a target (e.g., transcription factors, small molecules, peptides/protein fragments, proteins, lipids, nucleic acids, TCRs, kinases). In non-limiting embodiments, in vivo experiments can be constructed to identify an interaction between a library and an in vivo element (e.g., healthy, diseased, perturbed cells or tissues, and environmental conditions). In vitro experiments can also be constructed to identify an interaction between a library and an in vitro target (e.g., transcription factors, small molecules, peptides/protein fragments, proteins, lipids, nucleic acids).

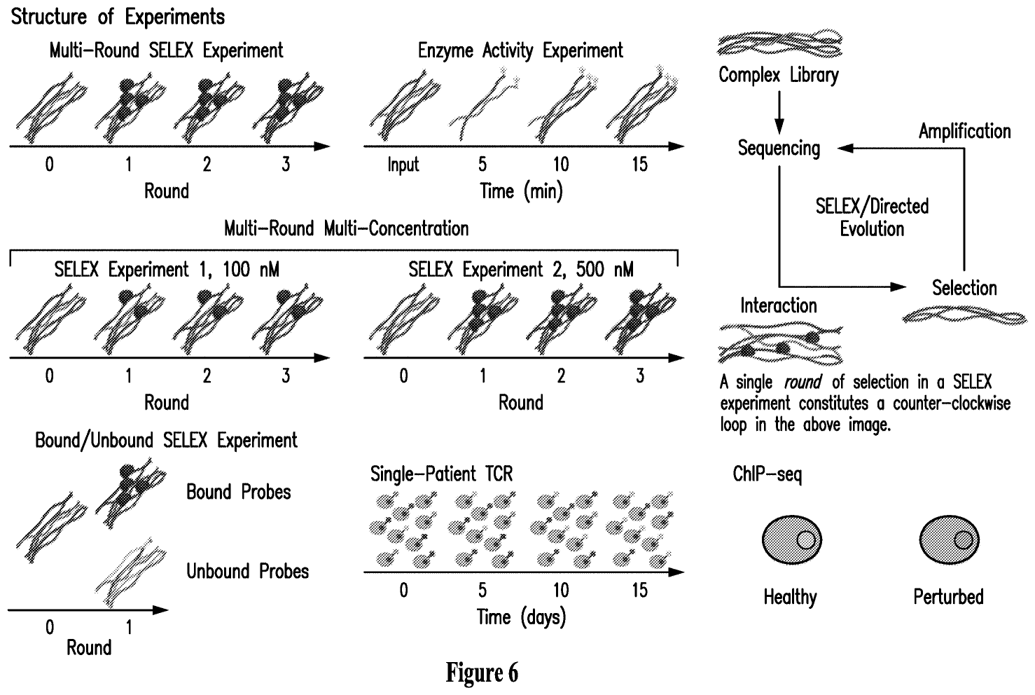

In non-limiting embodiments, the disclosed system can process various types of sequencing data generated from various sources and experimental designs. For example, as shown in FIG. 6, the disclosed system can process a single experiment, which can include at least two measurements taken at different times. The disclosed system can also analyze T-Cell repertories without using a random library or an in vitro binding experiment. Furthermore, the disclosed system can handle a genomic DNA sequence data for identifying protein-DNA binding activities between different environmental conditions. The disclosed system can also analyze cDNA library data to identify protein binding strength, binding K_{d}, enzymatic K_{M}, RNA binding K_{d}, DNA binding K_{d}, DNA binding strength in the presence of DNA methylation, or combinations thereof. In non-limiting embodiments, the disclosed system can process a plasmid sequence data generated from complex libraries for identifying TCR, pMHC binding strength, enzymatic activities, protein binding strength, or combinations thereof.

In certain embodiments, the disclosed data analytic platform 101 can generate the input data (e.g., high-throughput sequencing data) that can be utilized by the processor 102 to generate one or more evaluative models. The disclosed data analytic platform 101 can comprise a collection of hardware and software utilized to evaluate genetic data and generate the input data. For example, the data analytic platform 102 can include one or more high-throughput sequencing devices, PCR devices, bioinformatic software, and any other relevant and commonly used reagents, libraries, and other materials to evaluate sequence data. By way of non-limiting example, the data can be generated using various assay process (e.g., HT-SELEX, SELEX-seq, SMILE-seq, EpiSELEX-seq, CAP-SELEX, SPEC-seq, DAP-seq, Bind-n-seq, meHT-SELEX, NCAP-SELEX, RNA Bind-n-seq, HTR-SELEX, mRNA display, cDNA display, Ribosome display assays, ChIP-seq, ATAC-seq, DNase-seq, MPRAs, StaR-seq, SuRE-seq, TCR/BCR seq, RNA-seq, Hi-C, SPRITE, phage display, bacterial display, yeast display, Y1h pMHC display assays, or combinations thereof) and one or more high-throughput sequencing devices. In non-limiting embodiments, the input data can be generated from multiple libraries.

In certain embodiments, the data analytic platform 101 includes one or more data transmission devices that are utilized to output data to one or more local or remote data receivers, such as data processing platform 102. For example, the data analytic platform 101 can be configured to generate input data that can be directly transmitted to a computer or data processing platform 102. In non-limiting embodiments, the data generated by the data analytic platform 101 is stored or made accessible from a remote storage service, such as but not limited to, a commercial, private or custom cloud-based data hosting service or provider. In non-limiting embodiments, the input data generated by the data analytic platform 101 can be communicated to the processor 102 directly through one or more direct physical links, such as serial, RJ45, Parallel, USB, FIREWIRE, eSATA, fibreoptic, or other linkages.

Alternatively, the input data can be sent to a processor 102 that is physically remote from the data analytic platform 101. For example, the data can be transmitted wirelessly using one or more RF frequency-based communication protocols, such as WiFi, Bluetooth, or Zigbee protocols. In certain embodiments, the data analytic platform 101 can be configured to transmit data to the data processing platform 102 via a network connection or interfaces, such as a local intranet or the Internet. For example, the data analytic platform 101 can include one or more network adaptors that allow the data generated by the data analytic platform 101 to accessed remotely by the processor 102, or otherwise transmit data to the processor 102.

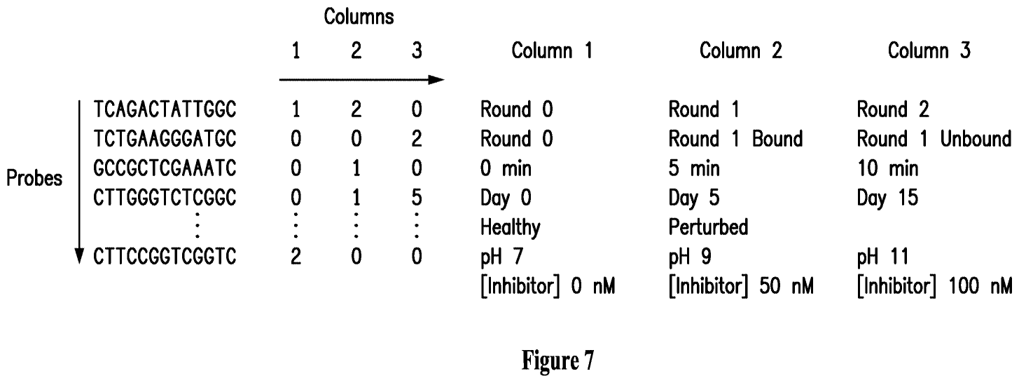

In certain embodiments, the disclosed subject matter includes generating an evaluative model based on the input data. In certain embodiments, the disclosed processor can receive, and the input data can compile the generated input data into a count table. As shown in FIG. 7, the count table can record the number of times that a unique sequence (i.e., rows of the count table) was observed in a particular round, time point, or condition in the experiments (columns of the count table). The observed sequence can be any general sequence including, but not limited to, DNA fragments of varying length, cDNA fragments of varying length, and BCR/TCR clonotype data. For example, the input data generated by each experiment can be summarized in the form of a count table K. For standard random DNA libraries whose design includes a variable region including L base pair positions that can be A, C, G, or T, the space of unique sequences (probes) can include all 4^{L} possible variants. These probes can define the rows of table K. Each particular library corresponds to a column of table K. The set of DNA reads obtained for a particular library C through massively parallel DNA sequencing can be summarized in the form of a read count vector K_{c} that can be interpreted as a multinomial sample from the probe space of size R_{c}, where R_{c} denotes the number of reads sequenced for the library C. The columns of table K can be grouped into experiments, which can be used for implementing constraints on the model that reflect the specific experimental design that was used to generate the data.

In non-limiting embodiments, the count table can be generated for non-standard DNA sequences. The count table can be used for methylated probes, RNAs, and proteins, which can include synthetic bases, unnatural base pairings, and unnatural amino acids. For example, the standard DNA sequences can include the four basic bases (e.g., A, C, G, or T). However, methylated probes can include bases with an alternative alphabet (e.g., A, mA, C, mC, G, mG, and T), and RNA sequences can also include different bases (e.g., A, C, G, and U). In non-limiting embodiments, the DNA sequences in the disclosed count table can represent molecules that are templated from DNA/RNAs (e.g., peptides generated using in vitro transcription and translation.

In non-limiting embodiments, multiple count tables corresponding to multiple experiments can be used to generate the evaluative model.

In certain embodiments, the evaluative model can be configured to acquire a biophysical model parameter, a model interaction parameter, a count table parameter, or combinations thereof utilizing the input data. In non-limiting embodiments, the evaluative model can also be configured to simultaneously use multiple biophysical models to represent one or more recognition modes of the biopolymers.

In certain embodiments, the disclosed processor can generate the evaluative model based on the input data. The processor 102 can include one or more computing elements (e.g., microprocessor or collections of microprocessors) that are configured to receive and evaluate data according to one or more instruction sets. For example, the processor 102 can be configured by a collection of modules, configured as code, circuits, or software, to implement certain functions and operations with respect to inputs received by one or more data processing devices.

In certain embodiments, the processor 102 is configured to receive data from the analytic platform 101 and process such received data using one or more model generating algorithms. The generated models can represent and/or characterize the relative affinities of all binding sites, from the optimal site all of the way down to those sites that are bound non-specifically. In non-limiting embodiments, a model generated using sequencing data can include data corresponding to the statistical distribution of the input data, the selection rate for each sequence of the input data, and binding affinity between the biopolymer and the target.

For example, and not by way of limitation, the processor 102 can evaluate the input data using one or more biophysical models of the recognition between the target molecule (e.g., transcription factors, small molecules, peptides/protein fragments, proteins, lipids, and nucleic acids) and the biopolymer library (e.g., DNA, RNA, methylated DNA, proteins including natural and/or unnatural amino acids) and data relating thereto and generates an analytical or evaluative model. In turn, this generated evaluative model can be used to probe, parse, and quantify the sequence-affinity relationship for a given target molecule across the full affinity range. The data that results from evaluating the target molecule-DNA/RNA/protein affinity using the evaluative model can be transmitted to a storage 104 or to the output device 105.

In certain embodiments, the disclosed processor can be configured to utilize a DNA/RNA/protein recognition model to evaluate the target molecule-DNA/RNA/protein affinity and interactions. In non-limiting embodiments, the processor can be configured to use the assumption that binding of the target molecule is affected by a DNA/RNA sequence over L base pairs, and thus is able to provide a model that maps each of the 4^{L} possible bound sequences S to a relative binding free energy ΔΔG(S)=ΔG(S)−ΔG(S0), where SO is a fixed reference sequence usually chosen to be the highest-affinity sequence. The corresponding relative binding affinity is given by Ka, rel=exp(−ΔΔG(S)/RT) with R denoting the ideal gas constant and T the absolute temperature. In non-limiting embodiments, a linear relationship can be assumed between the binding free energy and the various sequence features ϕ, that distinguish sequence S from the reference sequence. The recognition model can be:

$$\frac{Δ Δ G ( S )}{RT} = {\sum }_{ϕ} {β}_{ϕ} {X}_{ϕ} ( S ) . \qquad ( 1 )$$

βϕ represents the effect of each feature ϕ, on the binding free energy, as indicated by Xϕ(S), which equals 1 if S contains the feature and 0 if it does not. The set of features includes all possible mononucleotide substitutions (“mononucleotide model”). In certain embodiments, the dependencies among pairs of adjacent nucleotides (“dinucleotide features) can also be taken into account, such that both mononucleotide features and dinucleotide features can be incorporated into a single and/or a multi-mode model. In non-limiting embodiments, non-adjacent nucleotide interactions are also taken into account. For example, the disclosed system can consider higher-order interactions (e.g., trinucleotides). Thus, a resulting multi-mode model can be generated to provide improved predictive accuracy relative to the existing technological field, as such, an approach that better captures the effect of variation in DNA shape on the binding (e.g., effects of molecular structure on binding/activity).

In certain embodiments, the models generated according to the disclosed subject matter can incorporate more than a basic biophysical model of relative binding free energy. Binding of target molecule complexes at various offsets and/or orientations within a probe can contribute to its selection. In the absence of saturation, the frequency f1(S) of sequence S in the R1 library is proportional both to its frequency f0(S) in R0 and to the relative affinity with which it is bound. A non-limiting exemplary model can be:

$${f}_{1} ( S ) ∝ {f}_{0} ( S ) {\sum }_{v} [ {\sum }_{m} {e}^{Δ Δ {G}_{n s} ( {S}_{o} ) / RT} + {e}^{Δ Δ {G}_{n s}} / RT ] . \qquad ( 2 )$$

Here, Sv denotes the bound subsequence of length K for “view” v on the probe sequence of length L. The described approaches provide for a model that explicitly accounts for nonspecific binding (ΔΔGns/RT). Accounting for non-specific binding allows the resulting analytical model generated to achieve a more accurate prediction of relative affinities when compared to currently known and understood approaches. Moreover, since the same target molecule complex can bind DNA in different configurations, the model generated according to the described approach can be extended to a weighted sum over multiple recognition modes m in parallel. Finally, a multinomial distribution relates f1(S) to the observed (and unobserved) R1 counts of all 4^{L} unique sequences S. In this formalism, every unique sequence S can be considered its own “category.”

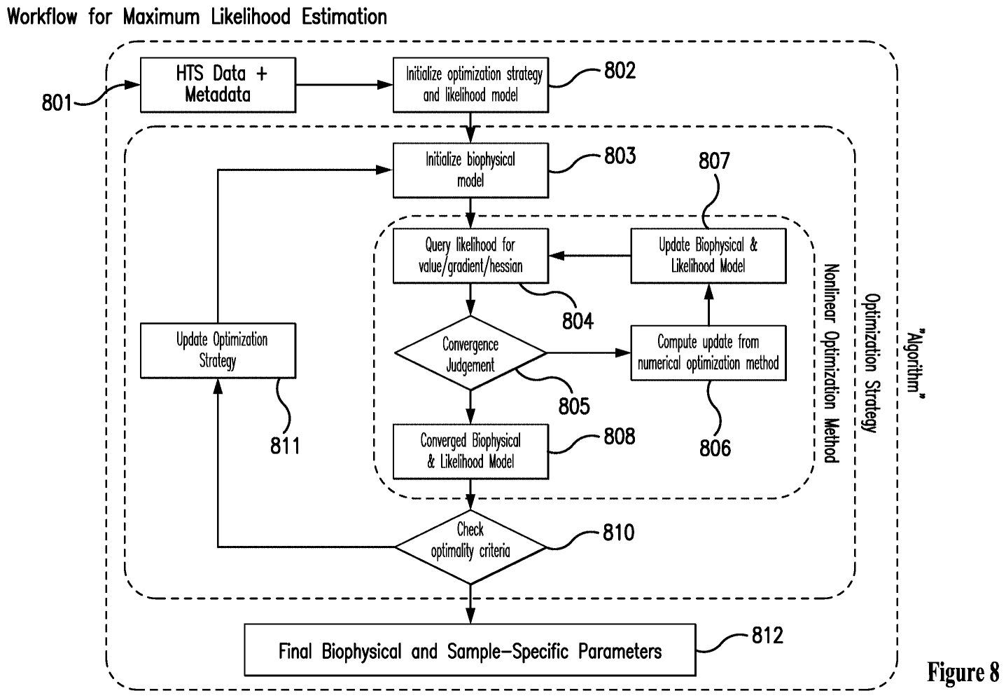

In certain embodiments, the evaluative model can include a biophysical model within a statistical framework where the parameters of the biophysical model can be determined using a maximum likelihood estimation approach. For example, the coefficients (e.g., βϕ and βns) of the DNA/RNA/protein sequence recognition model can be estimated using a likelihood maximization procedure that utilizes dedicated nonlinear optimization methods to make it feasible to fit the model in an efficient and robust manner. FIG. 8 provides a workflow of the maximum likelihood estimation (MLE) techniques 204. In certain embodiments, the disclosed model can be generated by optimizing the parameters of the biophysical model embedded in a statistical characterization of observed sequence data. For example, such a model can be generated as the composition of functions representing the statistical distribution of data (i.e., sequence data), the selection rate of the particular probe, and the bioactivity of the probe. The disclosed evaluative model can be optimized from at least one function representing a statistical distribution of the input data, a selection rate for each sequence of the input data, a binding affinity of the biopolymers, bioactivity of the biopolymers, an environmental condition of the biopolymers, a differential condition of biopolymers, or combinations thereof.

As shown in the flow diagram of FIG. 8, the processor 102 can be configured to implement a parameter optimization strategy to generate the evaluative model.

In certain embodiments, the processor 102 can utilize certain model fitting strategies to optimize the likelihood functions in a way that allows them to fit models without seeding, that is, without providing an initial likely value(s). For example, the processor can utilize a meta fitting strategy to increase the complexity of the model. In non-limiting embodiments, a model having optimized mononucleotide parameters can be used as a seed model for a model that includes nearest-neighbor dinucleotide features. For example, this model can be used as a seed model for one that includes all dinucleotides. In an alternative configuration, strategies focusing on shift symmetry and increasing the length of the model can be used to avoid incomplete models and capture an approximation of all sequence recognition specificity.

In non-limiting embodiments, the processor 102 can utilize a model fitting strategy that fits the model without approximating, truncating, or otherwise simplifying the objective function. In certain embodiments, the processor can utilize a model fitting strategy that fits the model without pre-processing the data. Such a model fitting strategy can be performed without filtering, aligning, or substring counting (kmer-counting) sub-process to fit the model.

For example, and not by way of limitation, the processor can initialize an optimization strategy and likelihood model 802 that incorporates the sequence data 801 obtained from the analytic platform (FIG. 8). The biophysical model can be initialized 803 as part of the optimization strategy. Once the initialized biophysical model is established, the processor can implement an optimization method to determine the optimal parameter set of the model. In non-limiting embodiments, the optimization method can be a nonlinear optimization method that evaluates the parameters using numerical optimization methods.

In certain embodiments, the non-linear optimization method can include conducting a query likelihood process for determining the value/gradient/hessian for the parameters 804 and evaluating the convergence of parameters of the biophysical model and the likelihood model 805. Where the models have not converged, updates to the model can be generated using numerical optimization 806. Such numerical optimization methods can include, but are not restricted to, zero-order methods (i.e., methods that utilize only the value of the likelihood) such as coordinate search or the Nelder-Mead simplex method, first-order methods (i.e., those that also utilize the gradient of the likelihood) such as gradient descent and stochastic gradient descent, quasi-newton methods (i.e., methods that utilize gradient information to approximate the hessian of the likelihood) such as the limited-memory BFGS method (L-BFGS) and its stochastic counterpart, stochastic L-BFGS, and second-order methods (i.e., those that utilize the hessian of the likelihood) such as Newton's method to optimize the parameters. The numerical optimization methods can be used to update the parameters of the biophysical model and likelihood model 807.

In certain embodiments, the disclosed processor 102 is configured to evaluate the converged models. For instance, the processor 102 can implement one or more functional analysis techniques in order to verify and ensure that converged models represent a true minima of the likelihood as opposed to a stationary point that is not a true representation of the minima. By way of further example, the model fitting strategies 804-807 can be implemented by the processor 102 to optimize the likelihood function in a manner that allows the model parameters to be optimized without seeding the models, approximating, or otherwise simplifying the objective function, or pre-processing the data.

In certain embodiments, the processor 102 can utilize a meta fitting strategy to increase the complexity of the model. By way of non-limiting example, a model having parameters optimized for mononucleotide mode interactions can be used as a seed model for a model that includes nearest-neighbor dinucleotide features, which in turn can be used as a seed model for a model that includes all dinucleotides. If the final goal of the converged model is to infer an all dinucleotide feature model, the optimizing process can begin by first inferring a mononucleotide model.

Once the mononucleotide model has converged, the inferred mononucleotide model can be used as a seed model for a more robust model that includes nearest-neighbor dinucleotide features. Once this model is optimized, the resulting optimized model can be used as a seed model to infer a model that includes all dinucleotides. In this manner, the disclosed processor 102 can generate a model that improves the fidelity of the model relative to prior, single recognition mode models. Through the use of the disclosed systems and methods, the time to fit large, complex models can be meaningfully reduced.

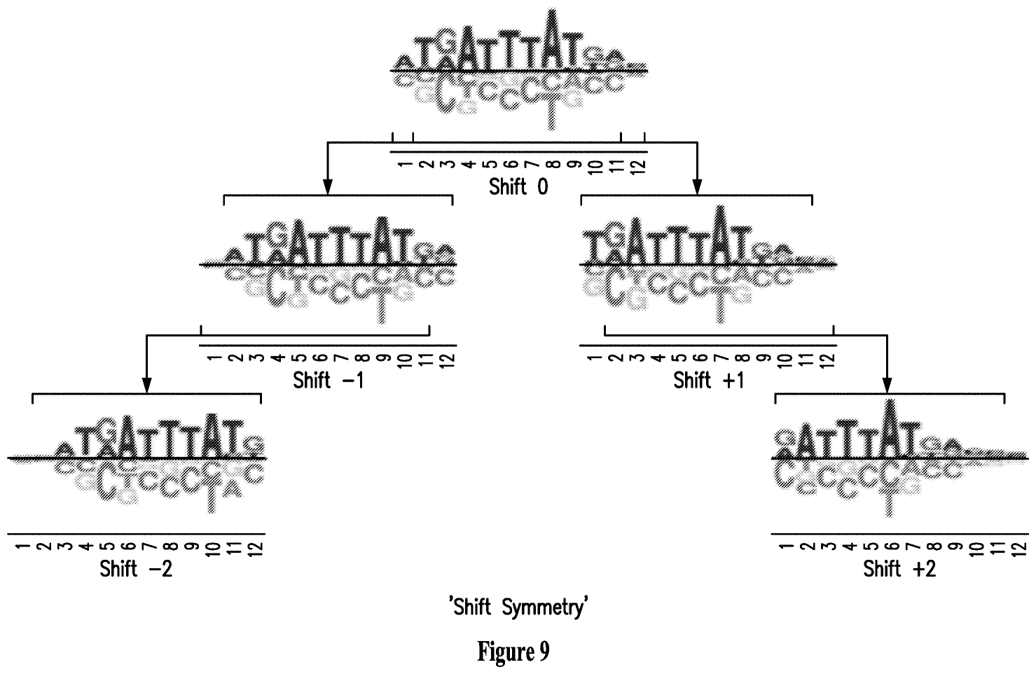

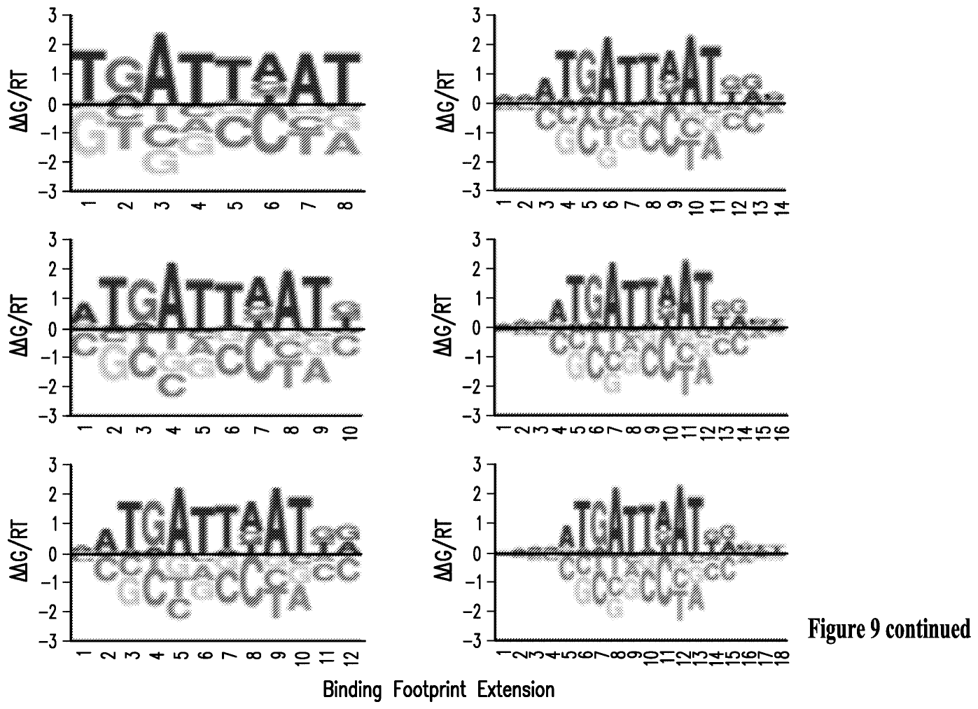

In certain embodiments, the use of additional recognition modes can also be used to increase the complexity of any given evaluative model. For example, and not by way of limitation, the disclosed subject matter can be used to evaluate individual recognition modes to determine whether the recognition mode is capturing all the binding specificity within an interface. For example, and not by way of limitation, the processor can be configured to increase the length of the recognition model and use one or more transformations, including, but not limited to, shift symmetry. As a result, the processor can generate a motif that can be ‘centered’ to capture nearly all of a transcription factor's binding specificity (FIG. 9). The presently disclosed system can be configured to avoid generating ‘incomplete’ models at any stage of the process. In non-limiting embodiments, strategies focusing on shift symmetry and increasing the length of the model can be used to avoid incomplete models and capture an approximation of all of the target molecule binding specificity (FIG. 9).

The updated models can be queried again for likelihood values. If, as shown in 808 in FIG. 8, there is convergence, the model parameters can be checked for optimality 810. Where optimality is present, the final biophysical and sequence-specific parameters can be output to the model as in 812. If optimality is not found, the process is continued such that the optimization strategy can be revised and updated 811. From here, the biophysical model can be re-initialized using at least one or more currently discovered model parameters and proceeds again to 810, as shown in the flow diagram of FIG. 8 until the optimality criteria are satisfied.

In certain embodiments, the generated model with the sample-specific parameters 812 can be utilized to identify biologically relevant binding sites in the biopolymer. In non-limiting embodiments, the generated model can be accurate and avoid further validation assays (e.g., EMSAs). In certain embodiments, the disclosed model can evaluate large footprint sizes, the disclosed subject matter can generate a model that can systematically analyze cooperativity for complexes of two or more biopolymers and target molecules. The multiple recognition mode functionality in the disclosed subject matter can provide a system that can utilize the disclosed model to capture both alternative complexes that can form within the same mixture of target molecules and alternative configurations (e.g., relative orientation, internal spacers) with which a given multi-target molecule complex can bind.

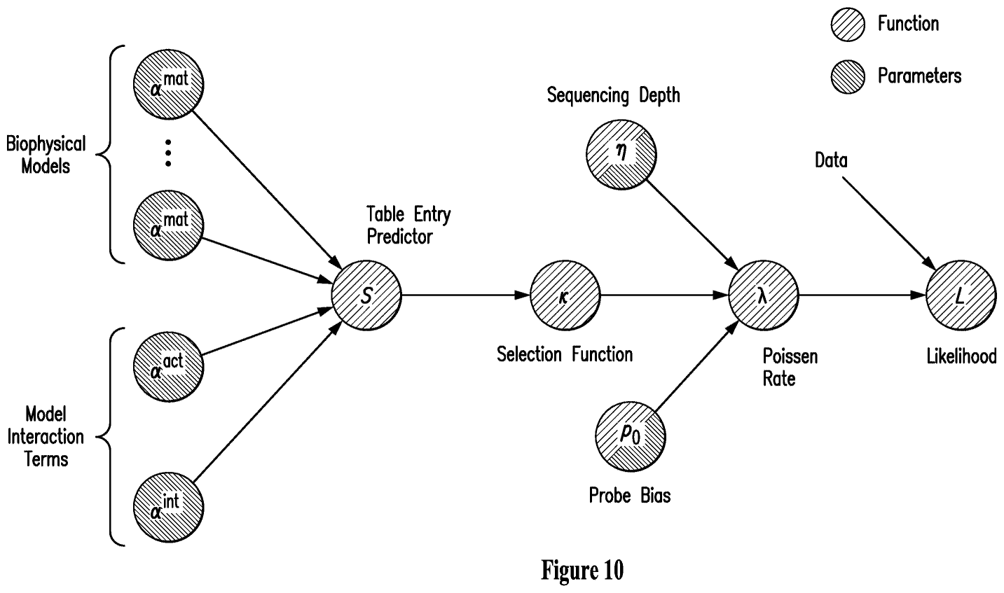

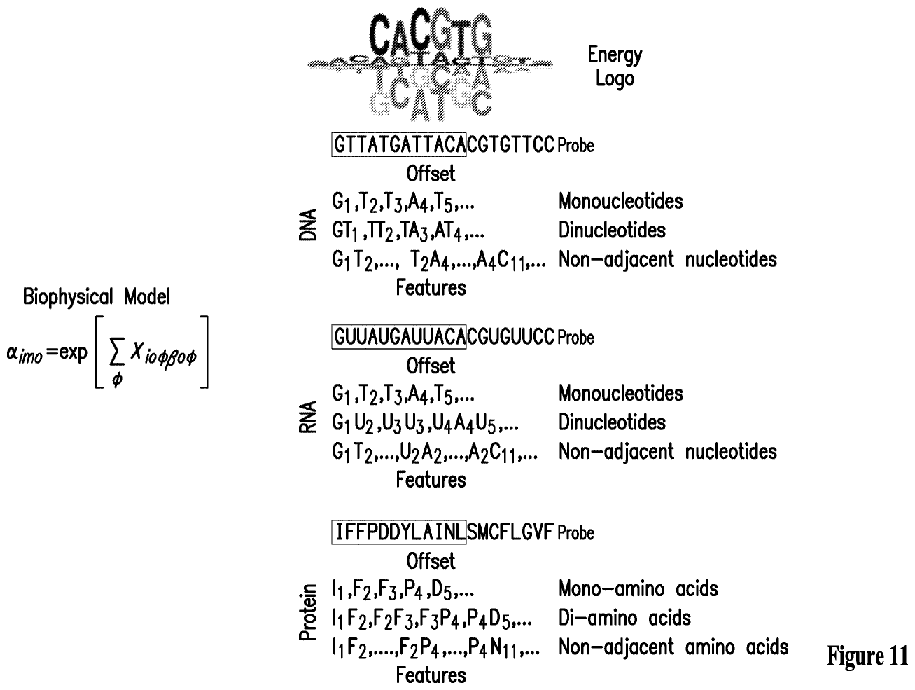

In certain embodiments, the disclosed evaluative model can utilize a biophysical model, a table entry predictor, a selection function, Poisson rate, a likelihood function, or combinations thereof to produce an improved and accurate evaluative model relative to existing models (FIG. 10). The disclosed evaluative model can be defined as a sum of generalized Poisson likelihood functions over count tables:

$$log ℒ ∝ {\sum }_{e ∈ E} {\sum }_{c ∈ {C}_{e}} {\sum }_{i ∈ {P}_{e}} {k}_{i , c , e} log {λ}_{i , c , e} - {λ}_{i , c , e} \qquad ( 3 )$$

where E is the set of experiments that are modeled, and e is a specific experiment in that set. Cₑ corresponds to all the columns in the count table associated with experiment e, while c is a specific column in the count table. Pₑ is the set of all probes in experiment e, and i is a specific probe sequence in this set. k_{i,c,e} is the count of probe i in count table columns c for experiment e, while λ_{i,c,e} is the modeled Poisson rate for the same probe and column.

In certain embodiments, the Poisson rate can be parameterized as follows:

$${λ}_{i , c , e} = {n}_{c , e} {p}_{i , 0} {κ}_{i , c , e} \qquad ( 4 )$$

Where η_{c,e} accounts for sequencing depth and other column-specific read-count biases, p_{i,0} models the probe bias in the input library, k_{i,c,e} models the selection of probe i in column c of experiment e. In non-limiting embodiments, analytically optimizing over η (i.e., requiring that ∂λ/∂η=0) can yield a likelihood function based on a multinomial distribution over probes i for each library (c, e). p_{i,0} can be modeled using round-zero (e.g., input library) data. In certain embodiments, analytically optimizing p_{i,0} can yield a likelihood that is based on multinomial distributions over libraries/samples for each probe, which no longer requires unobserved probes to be accounted for. The Poisson rate can be computed for every probe i by adjusting the selection function k by the inferred sequencing depth η and input probe p_{i,0}.

In certain embodiments, the disclosed modeling framework can allow various probe selection functions κ. The selection functions can be parameterized themselves in terms of ‘table entry predictor S_{i,c,e}. The expected selection rate of a probe λ can be driven by the probe-dependent selection function κ. The selection function can be dependent on table entry predictors, which can be any algebraic combination of probe sequence feature indicators and model parameters. The disclosed subject matter can provide various selection functions that can be highly flexible. Exemplary probe selection functions can be parameterized as follows:

$${κ}_{i , c , e} = {S}_{i , c , e} \qquad ( 5 )$$

According to the above equation, the probe selection function K corresponds to a simple linear selection model.

$${κ}_{i , c , e} = \frac{{( {S}_{i , c , e} )}^{{p}_{c , e}}}{{( 1 + {S}_{i , c , e} )}^{{γ}_{c , e}}} \qquad ( 6 )$$

According to the above equation, the probe selection function can be used in some K_{d} inference methods and model power-law dependent selection and/or binding saturation. γ_{c,e} and ρ_{c,e} correspond to column- and experiment-specific nonlinear enrichment.

$${κ}_{i , c , e} = {\prod }_{0 < {c}^{′} < c} \frac{{( {S}_{i , {c}^{′} , e} )}^{{ρ}_{{c}^{′} , e}}}{{( 1 + {S}_{i , {c}^{′} , e} )}^{{γ}_{{c}^{′} , e}}} \qquad ( 7 )$$

According to the above equation, the probe selection function can be used for the cumulative effect of power-law selection across multiple rounds.

$${κ}_{i , c , e} = {e}^{- {S}_{i , c , e}} or ( 1 - {e}^{- {S}_{i , c , e}} ) \qquad ( 8 )$$

According to the above equation, the probe selection function can be used to represent constant-rate, two-state kinetics. The first case can model exponentially decaying signals, while the latter case can model saturating signals. S_{i,c,e} corresponds to the product of the reaction rate and time.

$${κ}_{i , c , e} = {\vec{v}}_{o b s} \cdot {e}^{{\overleftrightarrow{S}}_{i , c , e}} \cdot {\vec{v}}_{i n i t} \qquad ( 9 )$$

According to the above equation, the probe selection function can be used to model kinetics in the disclosed system. \vec{ν}ᵢₙₜ is the initial state, \vec{ν}_{obs} is the observed state, and S_{i,c,e} is the exponentiated product of the transition rate matrix and time.

In certain embodiments, the disclosed models can utilize a table entry predictor. The table entry predictor S_{i,c,e} can be defined as scalars, vectors, matrices, or series of matrices that can be independently computed for combinations of every probe, column, and experiment using both the probe sequence (dependent only on i and e) and certain parameters dependent only on c and e. The table entry predictor can be any algebraic combination of probe sequence feature indicators and model parameters. In non-limiting embodiments, the table entry predictor can be composed of the sequence-specific readout from the biophysical models (α^{mat}) any interactions between these models (α^{int}) and some experiment and count table-specific biases (α^{act}).

For example, S_{i,c, e} is dependent on α^{mat}, or the free-energy prediction by the biophysical model m at offset o in probe i. In some embodiments, S_{i,c,e} can be dependent on α^{act} and α^{int}, a free parameters that correspond to the activity or interaction of recognition modes. In general, a table entry predictor can be any algebraic combination of the probe sequence feature indicators and model parameters. Exemplary quantities, not limited to, can be:

$${S}_{i , c , e} = {α}_{i , m , o}^{matrix} \qquad ( 10 )$$

According to the above equation, this table-entry predictor is for the specific case that a biomolecule can interact with the probe only at a specific offset.

$${S}_{ice} = {\sum }_{o} {α}_{imo}^{mat} \qquad ( 11 )$$

According to the above equation, this predictor corresponds to a ‘sliding window sum’ that the biophysical model α^{matrix} can be evaluated at every offset o in the probe.

$${S}_{ice} = {\sum }_{m} {\sum }_{o} {α}_{imo}^{mat} \qquad ( 12 )$$

According to the above equation, this predictor is extended from the above model to include ‘sliding window sums’ for multiple biophysical models (or recognition modes m).

$${S}_{i , c , e} = {\sum }_{m} {α}_{e , c , m , o}^{act} {\sum }_{o} {α}_{i , m , o}^{matrix} \qquad ( 13 )$$

According to the above equation, this predictor is extended from the above model to include α^{act}, a free parameter that corresponds to the activity of recognition mode m at offset o in column c of experiment e.

$${S}_{i , c , e} = {\sum }_{m} {α}_{e , c , m , o}^{act} {\sum }_{o} {α}_{i , m , o}^{matrix} + {\sum }_{{m}_{1} , {m}_{2}} {α}_{e , c , {m}_{1} , {m}_{2} , {o}_{1} , {o}_{2}}^{int} {\sum }_{{o}_{1} , {o}_{2}} {α}_{i , {m}_{1} , {o}_{1}}^{matrix} {α}_{i , {m}_{2} , {o}_{2}}^{matrix} \qquad ( 14 )$$

According to the above equation, this predictor is extended from the above model to include pairwise interaction (α^{act}) across all recognition modes.

In non-limiting embodiments, the table entry predictor can be modular and simultaneously model the impact of multiple modes. For example, and not by way of limitation, the probe selection function is able to model multiple recognition modes with mono- and all di-nucleotide features. In certain embodiments, one or more implementations, the table entry predictor can be configured to model the interactions between the models (i.e., mode interaction terms) as well as contributions from methylated and other chemically modified nucleotides. Furthermore, the table entry predictor can integrate the impact of all of the foregoing while also integrating multiple datasets and/or datasets that contain RNA data. As such, the presently described model can account for biases that occur in the process of obtaining the selected dataset (i.e., probe synthesis, double-stranding, and PCR amplification).

In certain embodiments, the disclosed models can utilize a biophysical model. The free-energy score of a biophysical model m, α^{mat}, corresponds to the “matrix score” of a “recognition mode” m at offset o. For example, α^{mat} is the biophysical model that represents the molecular interaction strength between a probe i and a recognition mode m at particular offset o in the probe. The score can be computed using the following biophysical model:

$${α}_{i , m , o}^{matrix} = exp [ {\sum }_{ϕ} {X}_{i , o , ϕ} {β}_{, ϕ} ] \qquad ( 15 )$$

where ϕ represents a feature of the sequence (e.g., monomer alphabet). The feature can include monomers present at different relative positions at offset and dimers made up of either neighboring or non-neighboring monomers. X_{i,o,ϕ} is a design matrix that specifies that features ϕ are present at offset o of probe i. β_{o,ϕ} parameterizes the impact of the different features on the total free-energy score. The disclosed biophysical model can use any algebraic combination of sequence features present.

In certain embodiments, the disclosed biophysical model is capable of fitting biophysical models that incorporate all mono-and di-nucleotide features present. Thus, the disclosed technique can be the approach capable of using a rich feature set in such a flexible and general way. For example, the disclosed system is able to utilize multiple recognition modes (m) represented in the data. In one or more implementations, the recognition model is able to use nearest-neighbor and/or incorporate all mono- and di-nucleotide features. These incorporated recognition modes can correspond to multiple configurations in which a target molecule binds to DNA, and/or multiple proteins present in a particular dataset. The disclosed biophysical model can use multiple recognition modes with various sequence feature sets.

In non-limiting embodiments, the disclosed biophysical model can fit with feature sets containing alternative alphabet data (e.g., methylated DNA, RNA, proteins, and unnatural amino acids). In non-limiting embodiments, the recognition model is able to use nearest-neighbor and/or incorporate all mono- and di- features with an alternative alphabet. In non-limiting embodiments, the recognition modes can also take into account the noise and/or technical artifacts present in the binding data. Likewise, the presently described model is able to represent unique recognition modes represented in the sequence data. These recognition modes correspond to multiple configurations in which a target molecule binds to DNA and/or multiple proteins present in a particular dataset, and/or noise/technical artifacts present in the data. Thus, the described model can utilize multiple recognition modes without the need for approximations.

In certain embodiments, the optimized parameter biophysical model can be used to provide, describe, or exemplify the molecular interactions of the target molecule binding affinity. As a non-limiting example, the foregoing disclosure describes utilizing a statistical model of sequence data that incorporates a biophysical model of protein-DNA binding affinity to provide actionable and useful data to identify protein binding sites within eukaryotic genomes or predict the functional impact of SNPs.

In certain embodiments, the optimized parameter biophysical model can be generated by modeling differences in selection rate for individual probes among two or more libraries. This statistical framework makes the approach described herein more general and versatile than approaches that utilize a single library. As a result of the approach described herein, there are no functional limits to the complexity of the biophysical models embedded inside the objective (or evaluative) function. Additionally, the multi-sample nature of the approach described herein allows for the evaluation of any type of in vivo, in vitro, or hybrid functional genomic data. Such analytical functionality represents an advancement over the prior approaches, which are limited to evaluating binding data using in vitro binding assays on random DNA libraries only.

In certain embodiments, certain constraints can be applied to the disclosed model to identify how the table-entry predictor S_{i, c, e} and the activity α^{act} are related across columns and experiments. Exemplary constraints can be:

$${S}_{i , c , e} = {[ P ]}_{free , c} {S}_{i , e} or {t}_{c} {S}_{i , e} \qquad ( 16 )$$

In the above equation, [P]_{free,c} is the free protein concentration in column c of experiment e, t_{c} is the treatment time of columns c, and S_{i,e} is constant across columns.

$${S}_{i , c , e} = {[ P ]}_{free , e} {S}_{i , c} or {t}_{e} {S}_{i , c} \qquad ( 17 )$$

In the above equation, [P]_{free,e} is the free protein concentration in experiment c, t_{c} is the treatment time of experiment e, and S_{i,c} is constant across experiments.

In the case where S_{i,c,e} is a linear function in activities, these constraints can be imposed for specific recognition modes or binding-mode interactions:

$${α}_{e , c , m , o}^{a c t} = {[ P ]}_{f r e e , c} {α}_{e , m , o}^{a c t} or {t}_{c} {α}_{e , m , o}^{a c t} \qquad ( 18 )$$

In the above equation, constraints are imposed for recognition mode m in experiment e across table columns.

$${α}_{e , c , m , o}^{a c t} = {[ P ]}_{f r e e , e} {α}_{c , m , o}^{a c t} or {t}_{e} {α}_{c , m , o}^{a c t} \qquad ( 19 )$$

In the above equation, constraints are imposed across experiments e. In non-limiting embodiments, the activity α^{act} can be independent from offset o and translationally invariant.

In certain embodiments, in computing the table-entry predictor S_{i,c.e} from the probe sequence, the sequence can be represented using an alphabet. The alphabet can be specified to be any set of characters. For example, A, C, G, and T can be used for standard DNA. A, C, G, U can be used for RNA. The single-letter amino acid codes can be used when analyzing proteins. Additional letters can be included to analyze, for example, chemically modified base pairs. In addition to the set of letters in the alphabet, the user can also specify complementary rules used to relate the sequences on the two strands of DNA. For example, the complementary rule can be A⇐⇒T, C⇐⇒G for standard DNA. Additional rules can be included to account for the complementary of chemically modified base pairs.

In certain embodiments, the processor can be configured to generate the evaluative models and evaluate genomic data using prior obtained source or training data. For example, the data values corresponding to the multiple selection round values for a given binding assay can be stored in the storage device. The processor data 102 can be configured to access and retrieve the values corresponding to multiple selection rounds of assay data from an accessible data storage location. In non-limiting embodiments, the processor 102 can be configured by one or more software modules to access data from one or more databases 104 that are accessible remotely from the data processing platform in response to a user-initiated query received from the remote computing device 105.

Alternatively, the processor 106 can be configured to receive current or contemporary assay and sequence data as part of the general workflow.

In certain embodiments, the processor can be configured to access and process multiple sequence data. For example, the processor can be configured to access the data corresponding to the sequenced nucleotides in the analytic platform 101 or the storage 104. Such an access process can include formatting data files, transmitting or retrieving data files, generating relevant queries to obtain the data, and other methods necessary to access and/or retrieve the data for use by the processor. Upon accessing the sequence data and obtaining a usable format thereof, the processor 102 can generate an analytical model based on the sequence data. For example, the processor 102 can be configured to utilize the formatted sequence data to generate a model that characterizes the relative affinities, binding free energy, kinetic rates, or a combination thereof of all binding sites.

In certain embodiments, the disclosed processor can be configured to estimate model parameters through a maximum likelihood estimation (MLE) approach that considers all possible binding sites within each ligand. Such an approach allows sensitive and accurate quantification of binding specificity over the full range (several orders of magnitude) of binding free energy, from optimal to nonspecific, without any prior information. Through embedding a biophysical model of target molecule-biopolymer interaction within a statistical model of the complete set of sequencing reads sampled in each experiment, the evaluative model can quantify target molecule-biopolymer interaction specificity.

In certain embodiments, the storage 104 can be a proprietary database that is accessed remotely using the interne or intranet and is operable as a remote computing platform (i.e., a cloud platform such as but not limited to Google, IBM, Azure, AWS, etc.) that permits access to and utilization of secure cloud computing services (e.g., data storage, on-demand GPU compute power, applications, etc.). In certain embodiments, the storage can contain data corresponding to multiple selection rounds (in SELEX, or other enrichment assays), the processor can access or establish a connection to the storage 104. Once a connection is established, the processor 102 can access and query the data from the database 104.

For instance, depending on the type and format of the data stored in the storage 104, the processor 102 can be configured to transmit one or more queries in SQL, NoSQL or another database schema to cause the database to return or transmit the requested data to the processing platform. In certain embodiments, the storage 104 can be configured as a local data storage device, such as a local hard disk, hard drive ROM, RAM, RAID array, storage cluster, or other types of data storage configuration commonly used in the art. In non-limiting embodiments, the processor 102 can provide a file or data structure navigator that allows a user to locate and access data stored in the respective data storage location.

In certain embodiments, the output device 104 can be a user terminal or computer that permits data exchanges with the processor 102. For instance, the output device can be one or more computers configured to connect to the processor 102 via a network connection. In non-limiting embodiments, the output device 104 can be configured with software that enables the bidirectional exchange of information with the processor 102. In further implementations, the output device 104 can be configured with standard software, such as a web browser, FTP, telnet, or other application that permits a user of the output device 104 to send instructions to the data processing platform and receive data in response thereto. In non-limiting embodiments, a user of the output device 104 can access the processor 102 using one or more user interfaces. The output device 104 is a local terminal that permits access to a local server or other computing platforms that provides the processor 102. In certain embodiments, the output device can be a remote terminal that communicates with the processor 102 over a wired or wireless network connection.

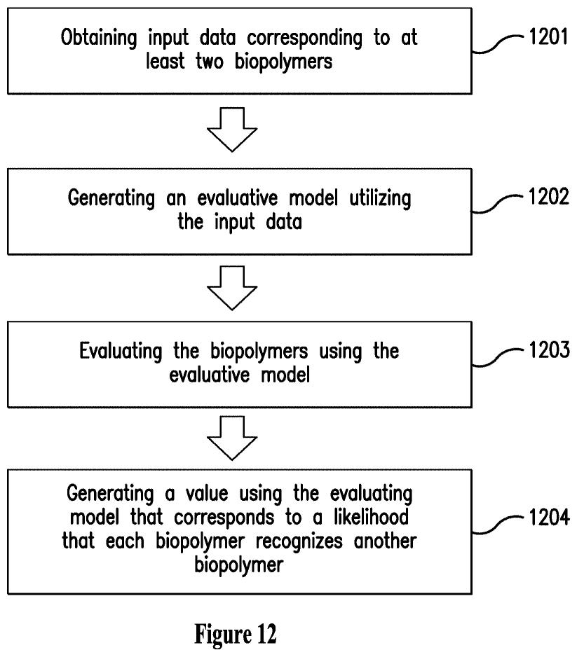

The presently disclosed subject matter also provides a method for determining binding preferences of a biopolymer library for a target molecule. The method can comprise obtaining input data corresponding to the biopolymers 1201, generating an evaluative model utilizing the input data 1202, evaluating the biopolymers using the evaluative model 1203, and generating a value using the evaluating model that corresponds to a likelihood that each biopolymer recognizes another biopolymer 1204 (FIG. 12). The evaluative model can be configured to acquire a biophysical model parameter, a model interaction parameter, a count table parameter, or combinations thereof from the input data, wherein the evaluative model is configured to simultaneously use multiple biophysical models to represent one or more recognition modes of the target molecule and the plurality of biopolymer libraries.

In certain embodiments, the disclosed method can include further processes for generating the input data. For example, and not by way of limitation, the disclosed method can further include obtaining a plurality of biopolymers, obtaining a first set of sequence data corresponding to sequence data for at least one of the plurality of biopolymers, exposing the plurality of biopolymers to a predetermined condition, obtaining a second set of sequence data corresponding to sequence data that for the biopolymers in the predetermined condition, and generating at least the first and second sets of sequence data as the input data for the evaluative model.

In certain embodiments, the method can include a further process for processing the input data. For example, and not by way of limitation, the disclosed method can further include compiling the input data into a count table. The count table includes a record of sequences of the biopolymers and a number of times that a probe of the biopolymer library is observed in an experimental condition.

In certain embodiments, the method can include further processes for optimizing the evaluative model. For example, and not by way of limitation, the disclosed method can further include optimizing the evaluative model using at least one function representing a statistical distribution of the input data, a selection rate for each sequence of the input data, a binding affinity of the biopolymers, bioactivity of the biopolymers, an environmental condition of the biopolymers, or combinations thereof.

In certain embodiments, the disclosed subject matter can be used to identify at least one Michaelis constant (K_{M}), dissociation constant (K_{d}), a presence of a putative binding site, a functional effect of a single nucleotide polymorphism (SNP), a transcription factor activity, a structural feature of a transcription factor, an immune response to a pathogen, thermostability, pH stability, protein binding strength, an enzymatic activity, a biopolymer interaction, antibiotic resistance, a difference between healthy and diseased cells, a cellular response to environmental variations, a regulatory pathway, an ability to penetrate a cell or tissue, or combinations thereof. In non-limiting embodiments, the disclosed subject matter can be used to generate a model based on bound/unbound for K_{d} estimation. In non-limiting embodiments, the disclosed subject matter can generate a model on Cas9 SEAM seq data. The generated model can build a complex, four-rate model directly from multiple time points of SEAM-seq data assaying Cas9 cleaving preferences.

In certain embodiments, the disclosed subject matter provides improved systems and methods for identifying bioactivities of biopolymers from sequence data of the biopolymers. The bioactivity can include binding affinity, binding free energy, interaction strength, kinetic rates, enzyme activity, antibiotic resistance, thermostability, a difference between healthy and diseased cells, cellular response to environmental variations, a regulatory pathway, an ability to penetrate a cell or tissue, a differential condition of biopolymers, or combinations thereof. In non-limiting embodiments, the bioactivity can be identified by inferring the coefficients of the disclosed recognition model using the high-throughput sequencing input data. The input data can include a sequence data library obtained from various custom-designed experiments for interaction between the disclosed target molecule and the disclosed biopolymer.

According to the disclosed subject matter, the probability of observing a particular read can be defined as a probe selection function that depends on the sequence of the read, the structure of the sequence recognition model, and on the numerical value of all relevant model parameters. Once the selection function is determined, the multinomial distribution over each library can be fully defined.

In certain embodiments, the probe selection function can be defined in multiple different ways. In non-limiting embodiments, the probe selection function can be defined in terms of an explicit mathematical expression (e.g., in the case of an equilibrium binding design where the parameters are used to predict binding energies associated with each sequence) or implicitly as the solution of a kinetic model in the form of a set of coupled differential equations (e.g., in the case of non-equilibrium binding assays or enzymatic assays, where the parameters P may be used to predict a given on-rate/off-rate/enzymatic rate for each sequence).

In certain embodiments, the disclosed subject matter can provide a flexible configuration of the objective function in terms of constraints. The disclosed multinomial likelihood can be a function of the sequence recognition model coefficients and any other parameters on which the selection function depends. In non-limiting embodiments, the disclosed multinomial likelihood can be further defined in terms of constraints that correspond directly to the experimental design, which was used to generate the data. For instance, in a multi-round SELEX-seq experiment, the coefficients should be the same in each round, as the same DNA-protein was used, but the parameters that account for variation in free protein concentration can be round-specific.

In certain embodiments, to estimate the model parameters from the input data, the disclosed subject matter can reformulate the maximum-likelihood inference model. For example, and not by way of limitation, the disclosed subject matter can consider a mathematically equivalent collection of multinomial distributions over all libraries for each unique probe sequence that is observed at least once in the dataset. As the set of unique probe sequences observed can be a minute fraction of the set of a probe that can be observed, this reformulation can be essential for making the inference computationally feasible. In non-limiting embodiments, alternatively, the disclosed subject matter can consider a collection of multinomial distributions over all possible unique probe sequences in each library, in which the parameters can be shared among all samples.

In certain embodiments, the disclosed subject matter can provide an accurate prediction of the effect in vivo/in vitro of DNA mutations on gene expression levels in organisms. Furthermore, the disclosed subject matter can provide an accurate prediction of synthetic sequences. Such predictions, in one implementation, are used to generate or engineer new sequences for use and analysis. For instance, based on the DNA-protein interface, predictions concerning binding interactions, the location of enhancer-binding sites, and the interpretation of gene regulation sequences can be made and evaluated without needing expensive or time-consuming validation binding assays. The disclosed subject matter can provide an improved level of predictive ability that holds true even in circumstances where the sequence mutation corresponds to very-low-affinity binding sites. Furthermore, such approaches allow for engineering sequences and determining the impact that such engineered sequences might have on gene expression levels.

In certain embodiments, the disclosed subject matter can provide a versatile maximum likelihood framework that can infer a biophysical model of the target molecule-biopolymer recognition across the full binding affinity range. The disclosed subject matter can overcome drawbacks and technical limitations in the field by being a pure computational approach that applies the rigorous analysis of data from experiments that use massively parallel DNA sequencing (high-throughput sequencing) to comprehensively probe protein-DNA interactions. In non-limiting embodiments, the disclosed subject matter can permit sequence data to be systematically interpreted in the context of personalized genomics, synthetic biology, and genetic engineering.

In certain embodiments, the disclosed subject matter can provide an improvement in the technological field. For example, the described techniques can predict human MAX homodimer binding in near-perfect agreement with existing low-throughput measurement. This technique can be more efficient, both in terms of computational cost as well as material and time, compared to existing techniques in the field. Furthermore, the disclosed subject matter can capture the DNA binding specificity of given proteins while distinguishing multiple recognition modes within a single sample. For example, the presently described approaches can simultaneously capture the binding specificity and distinguish the recognition modes related thereto using SELEX data.

In non-limiting embodiments, the presently described approaches can be used to confirm that newly identified low-affinity enhancer binding sites are functional in vivo, and that the contribution of the same to gene expression matches their predicted affinity. The disclosed subject matter can be used to identify new low-affinity enhancer binding sites and confirm that they are functional in vivo, with their contribution to gene expression matching their predicted affinity. Thus, the described approach established systems, methods, and computer products that set forth a powerful paradigm for identifying protein binding sites and interpreting gene regulatory sequences in eukaryotic genomes.

In certain embodiments, the disclosed subject matter also provides improved techniques to solve data sparsity. Certain DNA sequencing technology can handle a large number of sequence libraries. However, data sparsity (i.e., the fact that counts are low for many DNA sequences) can be a key limiting factor in the data analysis. Such sparsity can either result from the fact that the “space” of possible DNA sequences is extremely large (due to large genome size for in vivo data, or since the number of sequences in a random library grows exponentially with the length of the variable region for in vitro assays), or from an extreme degree of multiplexing (as in single-cell assays). To solve the data sparsity, the disclosed subject matter can avoid statistical analysis at the level of individual genes (e.g., differential expression detection in RNA-seq) or genomic loci (e.g., peak detection in ChIP-seq), or cells (e.g., classification by cell type in scRNA-seq), but rather use all the data to estimate global parameters that have biological meaning, such as feature-based binding energy contributions in the case of protein-DNA binding models, protein-level transcription factor activities, or selection signatures for specific epitopes in immune receptor repertoires.

### EXAMPLES

The following Examples are offered to illustrate the disclosed subject matter but are not to be construed as limiting the scope thereof.

### Example 1: Modeling for Single SELEX Experiment

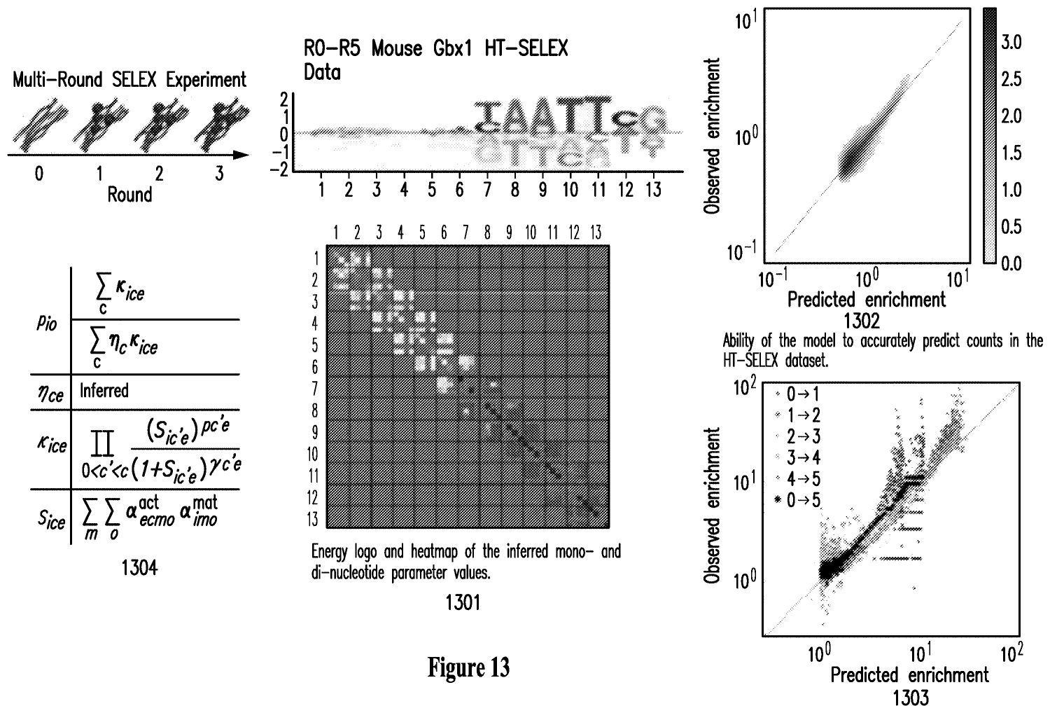

The disclosed subject matter was used to generate a model based on a single, multi-round HT-SELEX experiment assaying the DNA binding preferences of the mouse transcription factor Gbx1 (FIG. 13). Six libraries, consisting of the input round (R0) and five selection rounds (R1-R5), were used to train a model with mono- and di-nucleotide parameters. The model can be visualized using an energy logo and/or a heatmap 1301. The heatmap displays model mono- and di-nucleotide parameters in matrix format: the numbered rows and columns specify the position of the first and second base of the dinucleotide sequence feature within the binding site (diagonal blocks correspond to mononucleotides); within each row/column block, the four sub-columns and sub-rows correspond to “A,” “C,” G,” and “T.” The color of each cell represents the energy impact of each sequence feature: red indicates an increase in binding energy, blue indicates a decrease in binding energy, while white indicates no change; gray denotes parameters that, by definition, are zero. The model also accurately predicts counts in the HT-SELEX data set, as quantified by either comparing the observed and model-predicted k-mer (substring of length k) frequencies (scatterplot 1302) or by comparing the model predicted enrichment with the probe-count ratio between SELEX rounds (probes are first binned by model predicted affinity; count ratios are then computed for each bin; scatterplot 1303, the color corresponds the pair of SELEX rounds used to compute the enrichment as indicated by the legend). The mathematical details of the model are shown in 1304.

### Example 2: Modeling for Single SELEX Experiment

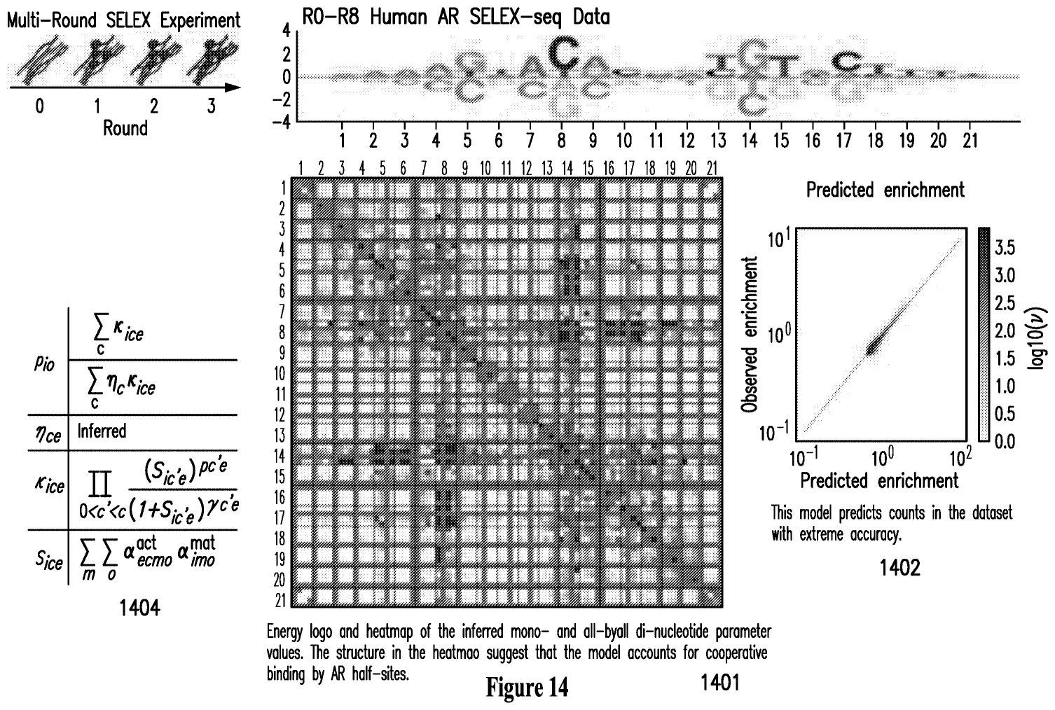

The disclosed subject matter was used to generate a model based on a single, multi-round SELEX-seq experiment assaying the DNA binding preferences of the human transcription factor AR (FIG. 14). Nine libraries, consisting of the input round (R0) and eight selection rounds (R1-R8), were used to train a model with mono- and all di-nucleotide parameters. The model can be visualized using an energy logo and/or a heatmap 1401. The structures in the heatmap suggest that the model accounts for cooperative binding by AR half-sites. The model also accurately predicts counts in the SELEX-seq dataset, as quantified by comparing the observed and model-predicted k-mer frequencies (scatterplot 1402). The mathematical details of the model are shown in 1403.

### Example 3: Modeling for Multi-Experiment Data

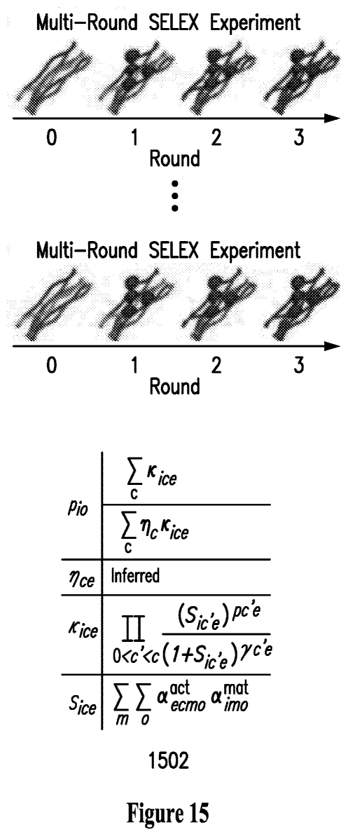

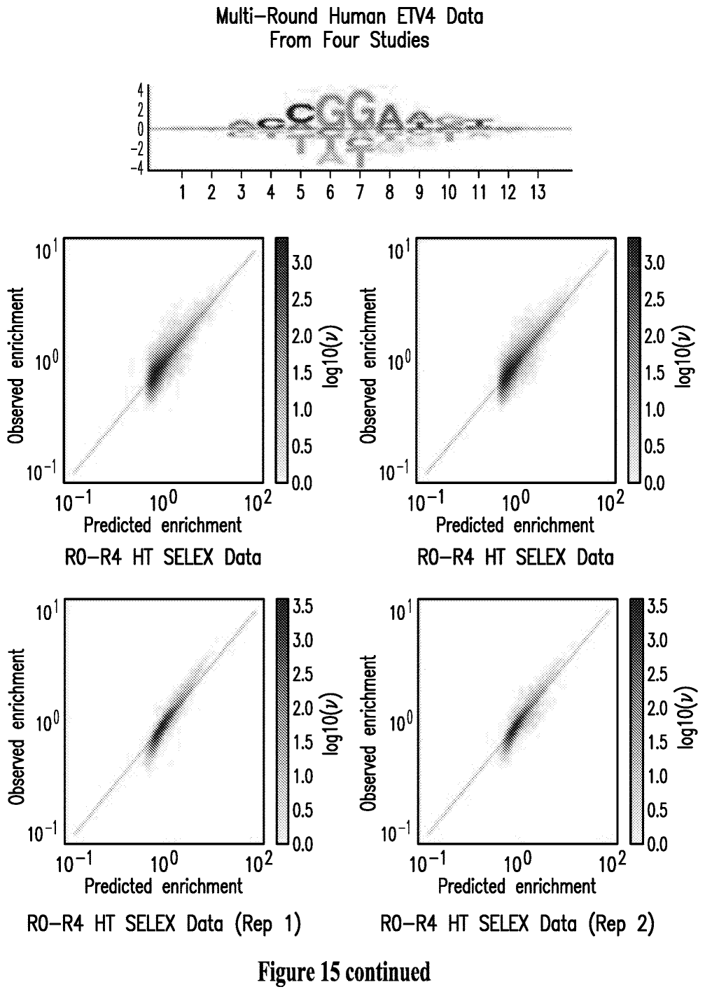

The disclosed subject matter was used to generate a model based on four independent, multi-round HT-SELEX experiments assaying the DNA binding preferences of the human transcription factor ETV4 is an example of multi-task learning (FIG. 15). Twenty libraries, five from each of the four experiments, were used to train a mononucleotide model, which can be visualized using an energy logo 1501. The model accurately predicts counts in all four HT-SELEX datasets, as quantified by comparing the observed and model-predicted k-mer frequencies (scatterplots 1501). The mathematical details of the model are shown in 1502.

### Example 4: Modeling for Multi-Experiment Data

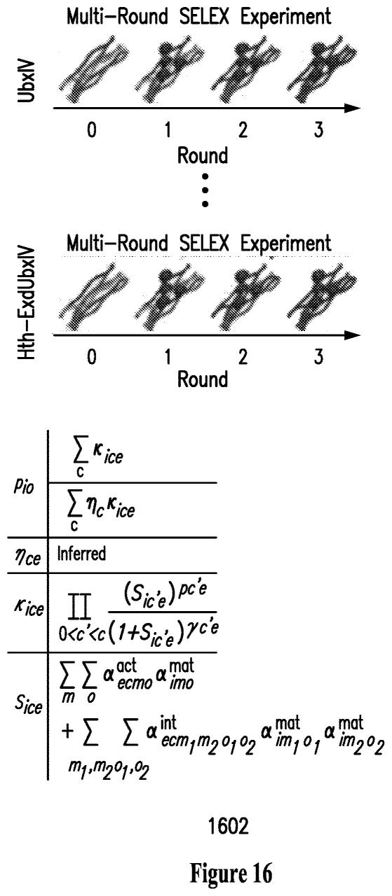

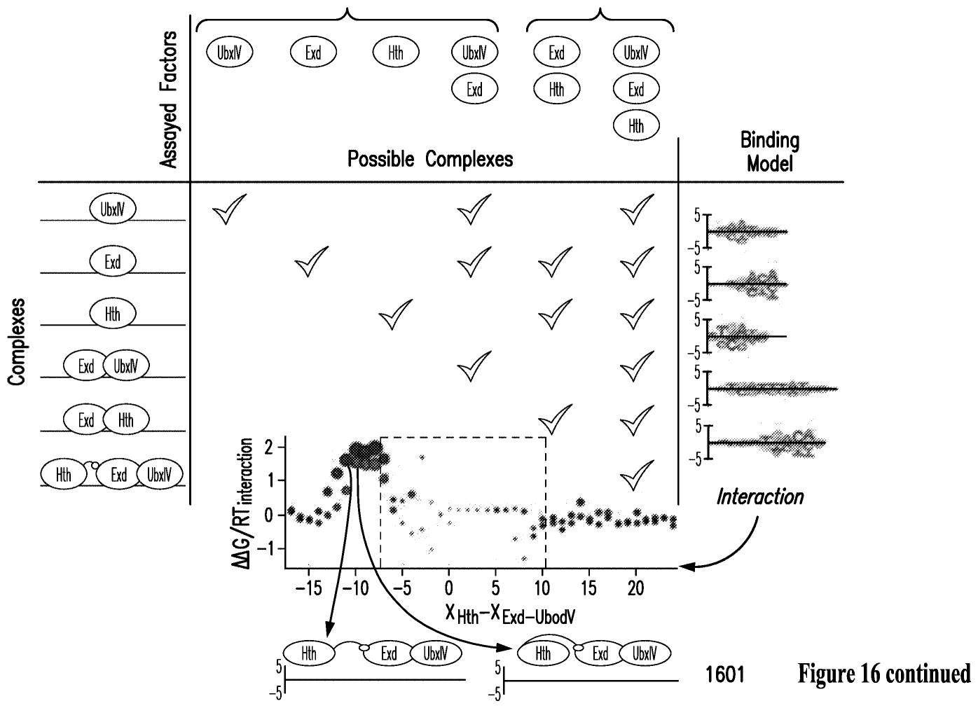

The disclosed subject matter was used to generate a model based on six independent, multi-round SELEX-seq experiments assaying the DNA binding preferences of three different Drosophila homeodomain transcription factors Hth, Exd, and UbxIV and their different complexes (FIG. 16). By assuming all possible (valid) permutations of complexes that can occur in the different experiments, a multiple sequence recognition mode model with mode interactions can be fit a model that was able to recapitulate known monomer and heterodimer sequence specificity and capture known heterotrimer spacing preferences 1601. The disclosed model can automatically discover these recognition modes and spacing preferences without the need for specialized computational analyses 1601. The experimental design (row headings) can be implemented as model constraints (green check marks indicating which binding modes participate in which experiment) 1601. The mathematical details of the model are shown in 1602.

### Example 5: Modeling for Methylated SELEX Data

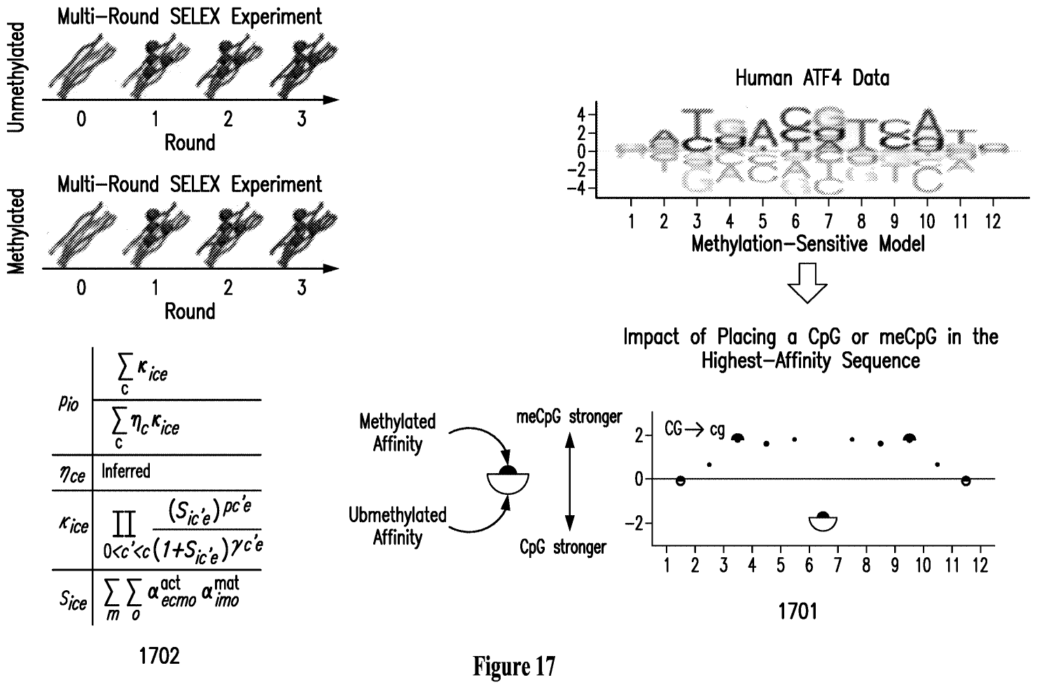

The disclosed subject matter was used to generate a model based on a methylated and an unmethylated single-round EpiSELEX-seq experiment assaying the DNA binding preferences and methylation sensitivity of the human transcription factor ATF4 (FIG. 17). Three EpiSELEX-seq libraries (i.e., input, methylated, and unmethylated) were used to simultaneously train a methylation-sensitive mononucleotide model, which can be visualized using an energy logo 1701, where the black lower-case ‘c’ and ‘g’ correspond to energy penalty of the meC-G and G-meC base pairs. 1701 also displays the energetic impact of a methylated (black half-circle) vs. unmethylated (white half-circle) CpG dinucleotides at any offset in the binding site. At a given position, the size of a half-circle is proportional to the relative affinity of a sequence containing a meCpG or CpG to that of the highest affinity sequence. The model was able to recapitulate the previously discovered methylation sensitivity of ATF4 automatically and without the need for specialized computational analyses. The mathematical details of the model are shown in 1702.

### Example 6: Modeling for K_{d} Estimation Based on Multi-Concentration Data

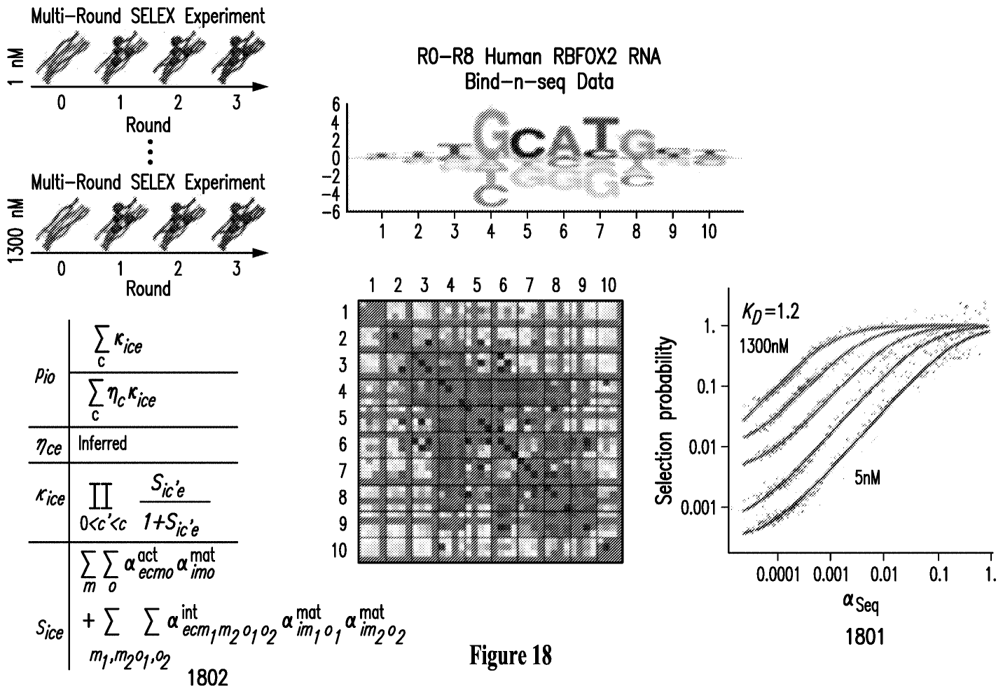

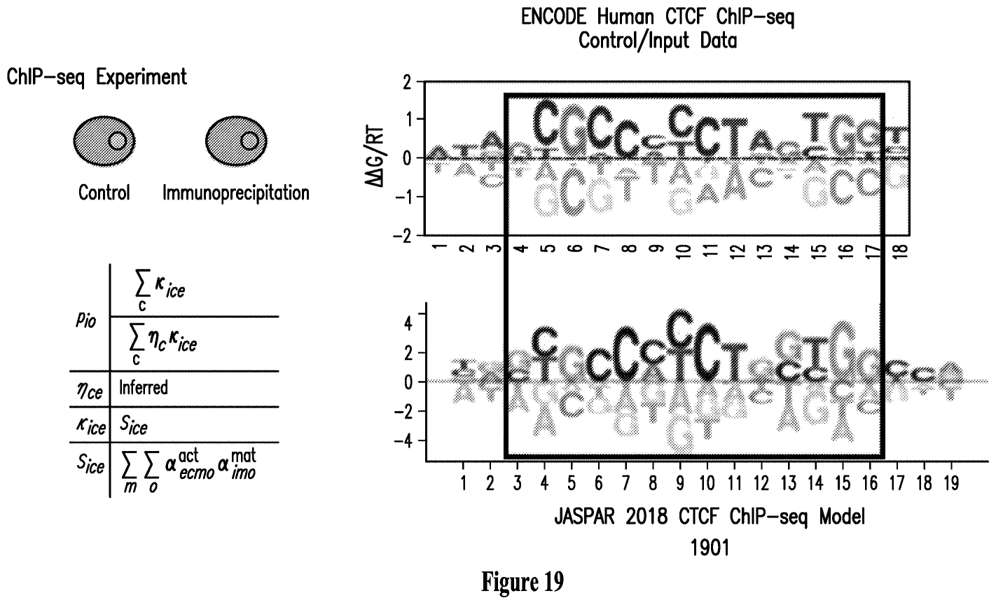

The disclosed subject matter was used to generate a model based on nine independent single-round RNA Bind-n-seq experiments at multiple concentrations (multi-concentration') assaying the RNA binding preferences of the human transcription factor RBFOX2 (FIG. 18). Ten libraries, including one input libraries and nine selection libraries at different concentrations, were used to train a mono- and all-dinucleotide model, which can be visualized using an energy logo and/or a heatmap 1801. Significantly, the model was able to correctly infer the optimal binding K_{d} of RBFOX2 directly from the

Bind-n-seq data, as shown by first plotting (for each concentration separately) the observed enrichment vs. the relative binding affinity (colored by the concentration of the dataset), then plotting the predicted enrichment vs. the relative binding affinity (for each concentration), and finally noting that the relative affinity at which binding saturation occurs agrees between these two plots. The mathematical details of the model are shown in 1802.

### Example 7: Modeling Based on ChIP-seq Data

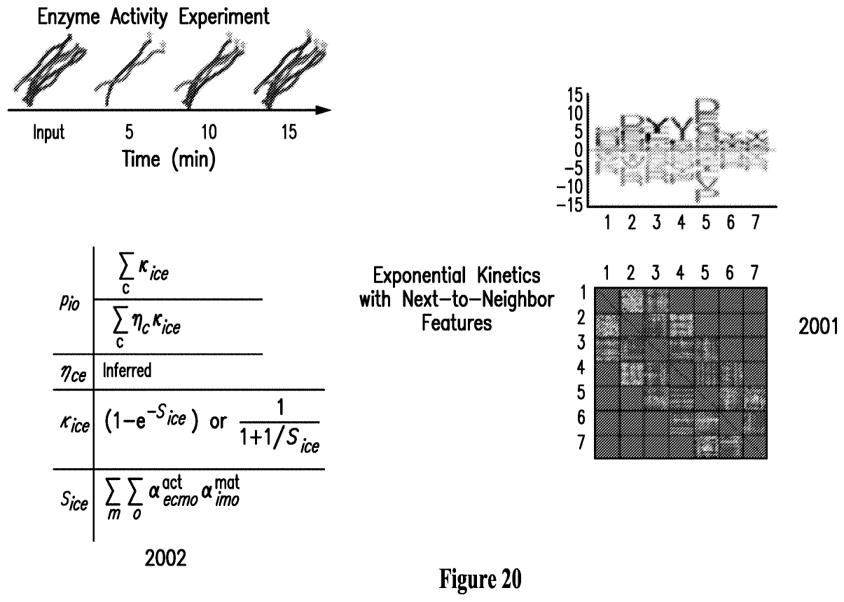

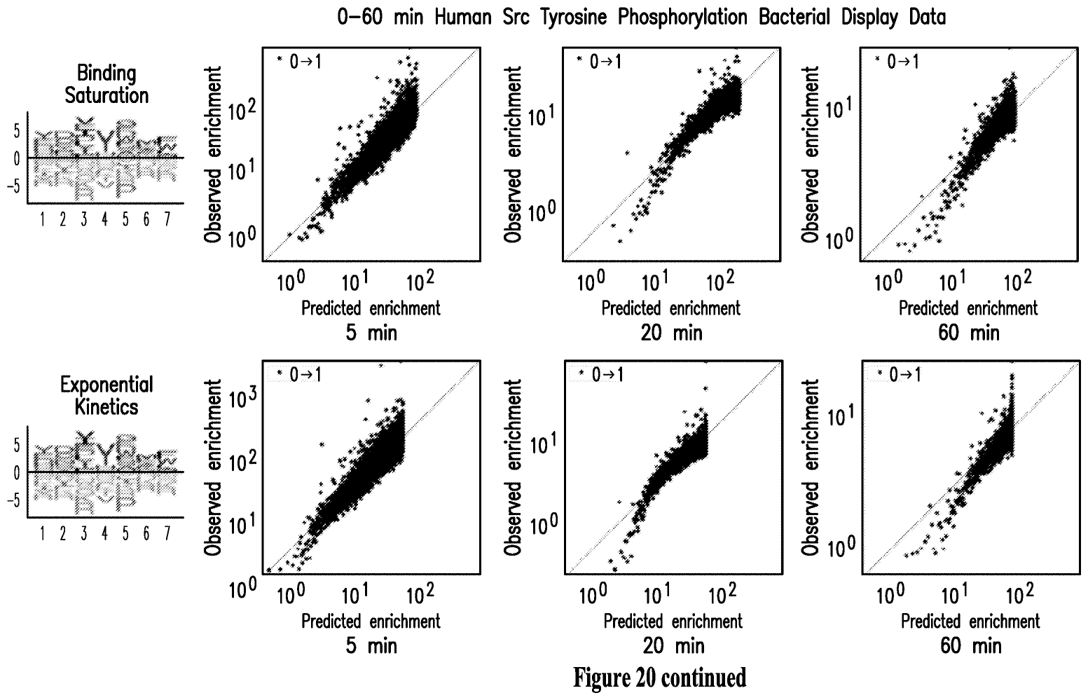

The disclosed subject matter was used to generate a model based on ChIP-seq data (FIG. 20). The first peak-free motif discovery model was created. Peaks are genomic regions where ‘significant enrichment’ of ChIP-seq reads occurred in the input dataset versus the control dataset; statistical methods such as MACS, SPP, etc. are used to identify such regions. A mononucleotide model for human CTCF trained on raw ENCODE ChIP-seq control/input data was able to accurately infer CTCF binding specificity when compared to the current ‘industry standard’ model from JASPAR, which was generated using the HOCOMOCO algorithm 1901. The HOCOMOCO algorithm fits models on post-processed ChIP-seq peaks. The mathematical details of the model are shown in 1902.

### Example 8: Modeling Based on Bacterial Display Data

The disclosed subject matter was used to generate a model based on bacterial display data (FIG. 20). The data was generated using a random library consisting of random polypeptides displayed on bacteria. Peptides phosphorylated by the human tyrosine kinase Src were isolated using a specific, high-affinity antibody. Using either a binding saturation or exponential kinetics model for x, the disclosed subject matter was capable of building mono-amino acid models capable of accurately modeling the three-time points of data. The generated model was capable of building a highly complex model with Next-Nearest-Neighbor features on the same data 2001. The mathematical details of the model are shown in 2002.

### Example 9: Modeling Based on Y1H pMHC Data

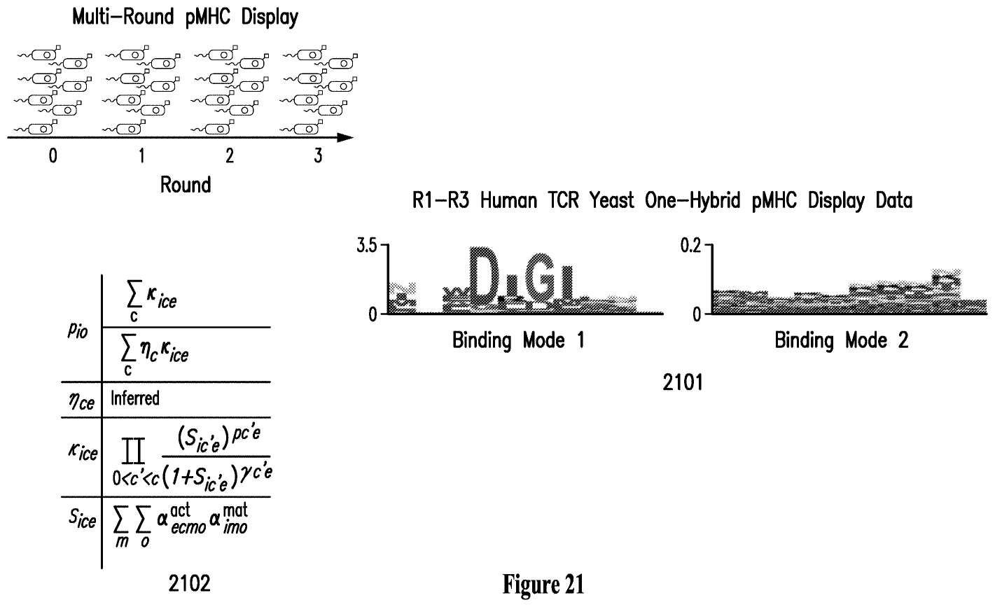

The disclosed subject matter was used to generate a model based on Y1H pMHC Data (FIG. 21). The human pMHC complex was used as the scaffold to construct the random library displayed on the surface of yeast. Affinity based selection was performed using bead-multimerized human TCR, thus profiling the pMHC specificity of the TCR of interest. The model was able to fit a two-mode mono amino-acid model using data generated after three rounds of affinity-based enrichment 2101. The details of the model are shown in 2102.

### Example 10: Modeling for Predicting Effect of SNP on Binding

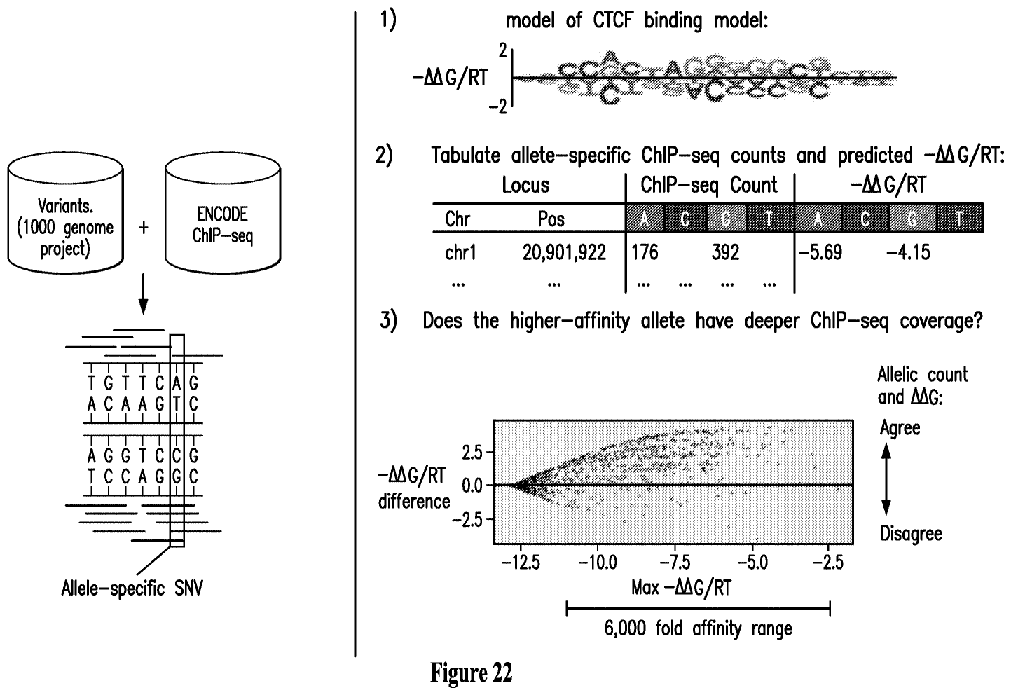

The disclosed subject matter was used to generate a model for identifying SNP (FIG. 22). The generated model was subsequently used to predict the effect of single-nucleotide polymorphisms (SNP) in the human genome. The predicted direction of change was in agreement with the observed change in genomic occupancy at the SNP location as measured by an in vivo allele-specific ChIP-seq assay over three orders of magnitude of affinity.

The features, structures, or characteristics of certain embodiments described throughout this specification can be combined in any suitable manner in one or more embodiments. For example, the usage of the phrases “certain embodiments,” “some embodiments,” “other embodiments,” or other similar languages, throughout this specification refers to the fact that a particular feature, structure, or characteristic described in connection with the embodiment can be included in at least one embodiment of the disclosed subject matter. Thus, the appearance of the phrases “in certain embodiments,” “in some embodiments,” “in other embodiments,” or other similar languages, throughout this specification does not necessarily refer to the same group of embodiments, and the described features, structures, or characteristics can be combined in any suitable manner in one or more embodiments. One having ordinary skill in the art will readily understand that the disclosed subject matter as discussed above can be practiced with procedures in a different order, and/or with hardware elements in configurations which are different than those which are disclosed. Therefore, although the disclosed subject matter has been described based upon these embodiments, it would be apparent to those of skill in the art that certain modifications, variations, and alternative constructions would be apparent while remaining within the spirit and scope of the disclosed subject matter.

What is claimed is:

1. A system for identifying bioactivities of biopolymers from sequence data of the biopolymers comprising :
an analytic platform configured to generate input data corresponding to the biopolymers;
a processor configured to receive input data; and
a storage medium coupled to the processor and comprising instructions operable when executed by the processors to cause the system to:
obtain the input data;
generate an evaluative model configured to acquire a biophysical model parameter, a model interaction parameter, a count table parameter, or combinations thereof utilizing the input data, wherein the evaluative model is configured to simultaneously use multiple biophysical models to represent one or more sequence recognition modes of the biopolymers;
evaluate the biopolymers using the evaluative model; and
generate a value using the evaluating model that corresponds to the bioactivity of each biopolymer.

2. The system of claim 1, wherein the biopolymers comprise a biopolymer library, wherein the biopolymer library includes a single-stranded deoxyribonucleic acid (DNA), double-stranded DNA, DNA with synthetic bases, DNA with unnatural base pairings, ribonucleic acid (RNA), RNA with synthetic bases, a protein with natural amino acids, a protein with unnatural amino acids, genomic DNA, methylated DNA, a fragment of genomic DNA, a plasmid, or combinations thereof.

3. The system of claim 1 further comprising an analytic platform configured to generate the input data corresponding to the biopolymers.

4. The system of claim 1, wherein the input data comprises at least two sets of biopolymer sequences, wherein the at least two sets of biopolymer sequences comprise a first set of biopolymer sequence data and a second set of biopolymer sequence data corresponding to a sequence of a biopolymer generated in the different conditions.

5. The system of claim 1, wherein the different conditions comprise an environmental condition, a disease state, a cell type or state, a tissue, a genotype, a presence or absence of a specific molecular target, a chemical modification, status of biopolymers, or a combination thereof.

6. The system of claim 1, wherein the input data is compiled into a count table, wherein the count table includes a record of sequences of the biopolymers and a number of times that a probe of the biopolymers is observed in the different conditions.

7. The system of claim 1, wherein the input data is stored in the storage medium.

8. The system of claim 1, wherein the evaluative model is optimized from at least one function representing a statistical distribution of the input data, a selection rate for each sequence of the input data, a binding affinity of the biopolymers, bioactivity of the biopolymers, an environmental condition of the biopolymers, or combinations thereof.

9. The system of claim 1, wherein the evaluative model is configured to generate the value corresponding to the log-likelihood using a sum of generalized Poisson log-likelihood functions over the count table.

10. The system of claim 9, wherein the sum of generalized Poisson log-likelihood functions over the count table is calculated based on sequencing depth, a probe bias in the input data, and a selection function.

11. The system of claim 10, wherein the sequencing depth, the probe bias in the input data, and the selection function are adjusted based on a target value to generate.

12. The system of claim 11, the target value is binding affinity, binding free energy, kinetic rate, or combinations thereof.

13. The system of claim 1, wherein the evaluative model is used to identify at least one Michaelis constant (K_{M}), dissociation constant (K_{d}), a presence of a putative binding site, a functional effect of single-nucleotide polymorphism (SNP), a transcription factor activity, a structural feature of a transcription factor, an immune response to a pathogen, thermostability, pH stability, protein binding strength, an enzymatic activity, a biopolymer interaction, antibiotic resistance, a difference between healthy and diseased cells, a cellular response to environmental variations, a regulatory pathway, an ability to penetrate a cell or tissue, or combinations thereof.

14. The system of claim 1 further comprising an output device configured to display the value.

15. A method for identifying bioactivities of biopolymers from sequence data of the biopolymers comprising:
obtaining input data corresponding to the biopolymers;
generating an evaluative model utilizing the input data, wherein the evaluative model is configured to acquire a biophysical model parameter, a model interaction parameter, a count table parameter, or combinations thereof from the input data, wherein the evaluative model is configured to simultaneously use multiple biophysical models to represent one or more sequence recognition modes of the biopolymers;
evaluating the biopolymers using the evaluative model; and
generating a value using the evaluating model that corresponds to the bioactivity of each biopolymer.

16. The method of claim 15, wherein the biopolymers comprises a biopolymer library, wherein the biopolymer library includes a single-stranded deoxyribonucleic acid (DNA), double-stranded DNA, DNA with synthetic bases, DNA with unnatural base pairings, ribonucleic acid (RNA), RNA with synthetic bases, a protein with natural amino acids, a protein with unnatural amino acids, genomic DNA, methylated DNA, a fragment of genomic DNA, a plasmid, or combinations thereof.

17. The method of claim 15 further comprising:
obtaining the biopolymers;
obtaining a first set of sequence data corresponding to sequence data for at least one of the biopolymers;
exposing the biopolymers to a predetermined condition;
obtaining a second set of sequence data corresponding to sequence data that for the biopolymers in the predetermined condition; and
generating at least the first and second sets of sequence data as the input data for the evaluative model.

18. The method of claim 15 further comprising compiling the input data into a count table, wherein the count table includes a record of sequences of the biopolymers and a number of times that a probe of the biopolymers is observed in the different conditions.

19. The method of claim 15 further comprising:
optimizing the evaluative model using at least one function representing a statistical distribution of the input data, a selection rate for each sequence of the input data, a binding affinity of the biopolymers, bioactivity of the biopolymers, an environmental condition of the biopolymers, or combinations thereof.

20. The method of claim 15, wherein the evaluative model is used to identify at least one Michaelis constant (K_{M}), dissociation constant (K_{d}), a presence of a putative binding site, a functional effect of single-nucleotide polymorphism (SNP), a transcription factor activity, a structural feature of a transcription factor, an immune response to a pathogen, thermostability, pH stability, protein binding strength, an enzymatic activity, a biopolymer interaction, antibiotic resistance, a difference between healthy and diseased cells, a cellular response to environmental variations, a regulatory pathway, an ability to penetrate a cell or tissue, or combinations thereof.
